(19) United States
(12) Patent Application Publication
(10) Pub. No.: US 2005/0075398 A1
(43) Pub. Date: Apr. 7, 2005

(54) Neuroprotectin D1 protects against cellular apoptosis, stroke damage, alzheimer's disease and retinal diseases

(76) Inventors: Nicolas G. Bazan, New Orleans, LA (US); Charles N. Serhan, Needham, MA (US); Victor L. Marcheselli, Covington, LA (US); Pranab K. Mukherjee, Metairie, LA (US); Sebastian G. Barreiro, Metairie, LA (US); Walter J. Lukiw, Slidell, LA (US); Song Hong, Brookline, MA (US); Karsten Gronert, New Fairfield, CT (US); Alberto E. Musto, New Orleans, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 10/911,835

(22) Filed: Aug. 5, 2004

Related U.S. Application Data
(60) Provisional application No. 60/493,110, filed on Aug. 5, 2003.
(60) Provisional application No. 60/564,426, filed on Apr. 22, 2004.
(60) Provisional application No. 60/589,445, filed on Jul. 20, 2004.

(51) Int. Cl. A61K 31/202

(52) U.S. Cl. 514/560

(57) ABSTRACT

A unique DHA product, 10, 17S-docosatriene (“Neuroprotectin D1” or “NPD1”), was found to provide surprisingly effective neuroprotection when administered right after an experimental stroke. Moreover, both nerve cells and retinal pigment epithelial (RPE) cells were found to synthesize 10,17S-docosatriene (NPD1) from DHA. NPD1 also potently counteracted H₂O₂/TNFα oxidative stress-mediated cell apoptotic damage. Under the same oxidative-stress conditions, NPD1 up-regulated the anti-apoptotic Bcl-2 proteins, Bcl-2 and Bcl-xL, and decreased expression of the pro-apoptotic proteins, Bad and Bax. Moreover, in RPE cells NPD1 inhibited oxidative stress-induced caspase-3 activation, IL-1β-stimulated human COX-2 promoter expression, and apoptosis due to N-retinylidene-N-retinylethanolamine (A2E). Overall, NPD1 protected both nerve and retinal pigment epithelial cells from cellular apoptosis and damage due to oxidative stress. NPD1 concentration in the brain of Alzheimer's patients was found to be significantly decreased from that of controls. In cultured human brain cells, NPD1 synthesis was up-regulated by neuroprotective soluble β amyloid, and NPD1 was found to inhibit secretion of toxic β amyloid peptides.

The benefit of the filing dates of provisional application 60/493,110 filed 5 Aug. 2003, 60/564,426 filed 22 Apr. 2004, and 60/589,445 filed 20 Jul. 2004 are claimed under 35 U.S.C. § 119(e) in the United States, and are claimed under applicable treaties and conventions in all countries.

This work was supported by National Institutes of Health grant nos. EY05121, NS23002, P20RR16816 from the COBRE Program, P01DE13499, and GM38765. The Government has certain rights in this technology.

### TECHNICAL FIELD

This invention pertains to the use of 10,17S-docosatriene (“neuroprotectin D1” or “NPD1”), a product derived from docosahexaenoic acid (DHA), to protect cells from apoptosis, to protect the brain from damage due to ischemic stroke, to help prevent Alzheimer's Disease, and to help prevent retinal degeneration.

### BACKGROUND ART

Docosanoids

Dietary omega-3 fatty acids are required to maintain cellular functional integrity, and overall are necessary to human health. Docosahexaenoic acid (22:6, n-3, DHA), a major component of fish oil and marine algae, is highly concentrated in photoreceptors, brain and retinal synapses. See, Bazan, N. G. (1990) in Nutrition and the Brain, vol. 8, eds. Wurtman, R. J. & Wurtman, J. J., (Raven Press, Ltd., New York) pp. 1-24. Diet-supplied DHA or its precursor (18:3, n-3) are initially taken up by the liver and then distributed through blood lipoproteins to meet the needs of organs, notably during photoreceptor cell biogenesis and synaptogenesis. See, Scott, B. L. & Bazan, N. G. (1989) Proc. Natl. Acad. Sci. U.S.A. 86:2903-2907. DHA has been reported to be involved in memory-related learning ability, excitable membrane function, photoreceptor cell biogenesis and function, and signal transduction pathways in which protein kinases are involved. See, Gamoh, S., Hashimoto, M., Sugioka, K., Shahdat Hossain, M., Hata, N., Misawa, Y., and Masumura, S. (1999) Neuroscience 93, 237-241; McGahon, B. M., Martin, D. S., Horrobin, D. F., and Lynch, M. A. (1999) Neuroscience 94, 305-314; Gordon, W. C., and Bazan, N. G. (1990) J. Neurosci. 10, 2190-2202; Mimikjoo, B., Brown, S. E., Seung Kim, H. F., Marangell, L. B., Sweatt, J. D., and Weeber, E. J. (2001) J. Biol. Chem. 276, 10888-10896. DHA has also been implicated in protecting nerve cells from apoptotic cell death as a membrane component, and other neuroprotective bioactivity. See, Kim, H.-Y., Akbar, M., Lau, A., and Edsall, L. (2000) J. Biol. Chem. 275, 35215-35223; Lauritzen, I., Blondeau, N., Heurteaux, C., Widmann, C., Romey, G., and Lazdunski, M. (2000) EMBO J. 19, 1784-1793; and Rodriguez de Turco, E. B., Belayev, L., Liu, Y., Busto, R., Parkins, N., Bazan, N. G., and Ginsberg, M. D. (2002) J. Neurochem. 83, 515-524. Whether DHA itself or a DHA-derived messenger is involved in these events is not known. DHA was also found decreased in the hippocampus of aged rats. (McGahon et al., 1999) Moreover, to date potent bioactive autacoids from DHA acting in nanomolar concentrations have not been identified in the central nervous system. Although certain docosanoids have been identified in retina, their physiologic properties have not been explored. See, Bazan, N. G., Birkle, D. L., and Reddy, T. S. (1984) Biochem. Biophys. Res. Commun. 125, 741-747; and Serhan, C. N., Clish, C. B., Brannon, J., Colgan, S. P., Chiang, N. & Gronert, K. (2000) J. Exp. Med. 192:1197-1204.

10,17S-docosatriene is a dihydroxy-containing DHA derivative. See, Hong, S., Gronert, K., Devchand, P. R., Moussignac, R. L. & Serhan, C. N. (2003) J. Biol. Chem. 278:14677-14687; and International Application No. WO 04/014835. Although the precise enzymes involved in NPD1 synthesis have not been identified, mounting evidence suggests that a PLA₂ enzymatic reaction which is then followed by a 15-lipoxygenase-like reaction is involved. PLA₂, which liberates free DHA from membrane phospholipids, and a 15-LOX-like activity, which converts DHA into NPD1 are prime candidates. See, FIGS. 3 and 11D; and Serhan, C. N., Clish, C. B., Brannon, J., Colgan, S., Chiang, N., and Gronert, K. (2000) J. Exp. Med., 192, 1197-1204; Serhan, C. N., Hong, S., Gronert, K., Colgan, S. P., Devchand, P. R., Mirick, G., and Moussignac, R. L. (2002) J. Exp. Med 196, 1025-1037; and Hong, S., Gronert, K., Devchand, P. R., Moussignac, R. L., and Serhan, C. N. (2003) J. Biol. Chem. 278, 14677-14687.

Neuronal Damage During Ischemic Stroke

Brain ischemia-reperfusion triggers lipid peroxidation that participates in neural injury. See, Bazan, N. G., and Allan, G. (1998) In Cerebrovascular Disease: Pathophysiology, Diagnosis, and Management, eds. Ginsberg, M. D. and Bogousslavsky, J. (Blackwell Science, Inc., Malden, Mass.) pp. 532-555 Docosahexaenoate (22:6n-3, DHA), esterified in membrane phospholipids, is released in brain ischemia, and is thought to yield lipid peroxides. See, Bazan, N. G. (1970) Biochim. Biophys. Acta 218, 1-10; Yoshida, S., Harik, S., Busto, R., Santiso, M., Martinez, E., and Ginsberg, M. D. (1984) J. Neurochem. 42, 711-717; and Roberts, L. J. II, Montine, T. J., Markesbery, W. R., Tappert, A. R., Hardy, P., Chemtob, S., Dettbarn, W. D., and Morrow, J. D. (1998) J. Biol. Chem. 273, 13605-13612 Leukocyte infiltration and pro-inflammatory gene expression are mediators of ischemic stroke damage; however, no messengers that modulate these events are known. See, Rothwell, N. J., and Luheshi, G. N. (2000) Trends Neurosci. 23, 618-625; Matsuo, Y., Onodera, H., Shiga, Y., Nakamura, M., Ninomiya, M., Kihara, T., and Kogure, K. (1994) Stroke 25, 1469-1475; and Royo, N. C., Wahl, F., and Stutzmann, J.-M. (1999) NeuroReport 10, 1363-1367. The biosynthesis of oxygenated arachidonic acid messengers triggered by cerebral ischemia-reperfusion is preceded by an early and rapid phospholipase A₂ activation reflected in free arachidonic and docosahexaenoic acid accumulation. See, Gaudet, R. J., and Levine, L. (1980) Stroke 11, 648-652; Moskovitz, M. A., Kiwak, K. J., Hekimian, K., Levine, L. (1984) Science 224, 886-899; Bazan, N. G. (1970) Biochim. Biophys. Acta, 218, 1-10; Yoshida, S., Harik, S., Busto, R., Santiso, M., Martinez, E., and Ginsberg, M. D. (1984) J. Neurochem. 42, 711-717; and Aveldano, M. I., and Bazan, N. G. (1975) Brain Res. 100, 99-110. These fatty acids are released from membrane phospholipids where they are esterified. See, Bazan, N. G. (1970) Biochim. Biophys. Acta 218, 1-10. Both fatty acids are derived from dietary essential fatty acids; however, only DHA is concentrated in the central nervous system. See, Bazan, N. G. (1990) in Nutrition and the Brain Vol. 8, eds. Wurtman, R. J. and Wurtman, J. J. (Raven Press, Ltd., New York) pp. 1-24. It is clear that synaptic membrane and retinal photoreceptor biogenesis is dependent on liver processing of the dietary DHA or of its precursor linolenic acid, followed by blood lipoprotein transport. See, Scott, B. L., and Bazan, N. G. (1989) Proc. Nat. Acad. Sci. USA, 86, 2903-2907.

Alzheimer's Disease

Inflammatory pathways participate in the maintenance and progression of Alzheimer's disease (AD) neurodegeneration. Cytokine-mediated oxidative stress, the accumulation of reactive oxygen species (ROS) and complement proteins, pro-inflammatory mediators and the activation of microglia associated with Aβ peptides and neuritic plaques confer a state of sustained and progressive inflammation in AD-afflicted brain. See, McGeer, P. L., and McGeer, E. G. (1999) J Leukoc. Biol., 65: 409-15; McGeer E. G., and McGeer P. L. (2003) Prog. Neuropsycho-pharmacol. Biol. Psychiatry., 27: 741-749; Lukiw, W. J., Bazan, N. G. (2000) Neurochem. Res., 25:1173-1184; Hoozemans, J., Veerhuis, R., Rozemuller A, Eikelenboom, P. (2002) Drugs Today, 38: 429-443; Zhang, R., Brennan, M. L., Shen, Z., MacPherson, J. C., Schmitt, D., Molenda, C. E., Hazen, S. L. (2002) J. Biol. Chem., 277: 46116-46122; and Casserly, I., Topol, E. (2004) Lancet, 363: 1139-1146. Free radicals and pro-inflammatory cytokines, e.g., such as interleukin-1beta (IL-1β), activate both beta amyloid precursor protein (βAPP) gene transcription and cyclooxygenase-2 (COX-2) in AD as well as in brain cells in culture. See, Bazan, N. G., Colangelo, V., Lukiw, W. J. (2002) Prostaglandins and Other Lipid Mediators, 68:197-210; Bazan, N. G., Lukiw, W. J. (2002) J. Biol. Chem., 277:30359-30367; and Lahiri, D. K., Chen, D., Vivien, D., Ge, Y. W., Greig, N. H. (2003) J Alzheimer's Dis., 5:81-90.

sAPPα is the 612-amino acid fragment derived from α-secretase-mediated cleavage of βAPP and is reported to possess neurotrophic activities. See, Selkoe, D. J. (2004) Ann. Intern. Med., 140: 627-638. Importantly, the sAPPα-generating α-secretase pathway does not give rise to the shorter amyloidogenic Aβ peptides. Hence the shunting of βAPP into the α-secretase pathway may have a beneficial effect, in part, by the relative lowering of neurotoxic Aβ peptide levels. sAPPα supports neuritogenesis and normal neuronal cell signaling function and protects primary neurons from the toxicity of Aβ40 and Aβ42 peptides. See, Selkoe, 2004. sAPPα also promotes long-term survival of hippocampal and cortical neurons in culture and protects brain cells against excitotoxic and ischemic injury in cell cultures and in vivo. See, Cheng, G., Yu, Z., Zhou, D., Mattson, M. P. (2002) Exp. Neurol. 175: 407-14.

Alzheimer's patients are known to have a low serum concentration of DHA. Low levels of DHA in the brain are associated with onset of Alzheimer's Disease and cognitive decline during aging. See, Farooqu, A. A., Horrocks, L. A. (2001) J. Mol. Neurosci. 16: 263-72; and Puskas, L. G., Kitajka, K., Nyakas, C., Barcelo-Coblijn, G., and Farkas, T. (2003) Proc. Natl. Acad. Sci. 100: 1580-1585.

Retinal Pigment Epithelial Cells and Retinal Diseases

Photoreceptor outer segments contain rhodopsin as well as the highest content of DHA of any cell type. In contact with the photoreceptor tips is a monolayer of cells, the retinal pigment epithelium (RPE), derived from neuroepithelium. These cells are the most active phagocytes of the body. In a daily cycle, they engulf and phagocytize the distal tips of photoreceptor outer segments, thereby participating in rod outer segment renewal in a process that is balanced by addition of new membrane to the base of the outer segments. The conservation of DHA in photoreceptors is supported by retrieval through the interphotoreceptor matrix, which supplies the fatty acid for the biogenesis of outer segments. See, Stinson, A. M., Wiegand, R. D. & Anderson, R. E. (1991) J Lipid Res. 32:2009-2017; Bazan, N. G., Birkle, D. L. & Reddy, T. S. (1985) in Retinal Degeneration: Experimental and Clinical Studies. Eds. LaVail, M. M., Anderson, R. E., & Hollyfield, J. (Alan R. Liss, Inc., New York) pp. 159-187; and Gordon, W. C., Rodriguez de Turco, E. B. & Bazan, N. G. (1992) Curr. Eye Res. 11:73-83. The continuous renewal of photoreceptors is tightly regulated so that their length and chemical composition, including that of their phospholipids, are maintained. Photoreceptor phospholipids contain most of their DHA in carbon 2 of the glycerol backbone. However, they also display molecular species of phospholipids containing DHA in both C1 and C2 positions of the glycerol backbone, as well as polyunsaturated fatty acids of longer chains than C22 that result from subsequent elongation of DHA. See, Choe, H-G & Anderson, R. E. (1990) Exp. Eye Res. 51:159-165. Retina, as well as brain, displays an unusual DHA-retention ability, even during very prolonged dietary deprivation of essential fatty acids of the omega-3 family. In fact, to effectively reduce the content of DHA in retina and brain in rodents and even in non-human primates, dietary deprivation for more than one generation has been necessary. Under these conditions, impairments of retinal function have been reported. See, Wheeler, T. G., Benolken, R. M. & Anderson, R. E. (1975) Science. 188:1312-1314; and Neuringer, M., Connor, W. E., Van Petten, C. & Barstad, L. (1984) J. Clin. Invest. 73:272-276.

DHA is highly concentrated as an acyl group of phospholipids in photoreceptor outer segment disc membranes. The RPE cell actively recycles DHA from phagocytized disc membranes back to the inner segment of the photoreceptor cell. In addition, the RPE cell takes up DHA from the blood stream through the choriocapillaris. The RPE cell thus is very active in the uptake, conservation, and delivery of DHA.

The high content of DHA in photoreceptor and RPE cells has to date been linked mainly to endowing photoreceptor membrane domains with physical properties that contribute to the modulation of receptors (e.g., rhodopsin), ion channels, transporters, etc. For example, in other cells DHA modulates G-protein-coupled receptors and ion channels. Moreover, DHA has been suggested to regulate membrane function by maintaining its concentration in phosphatidylserine. See, Salem, N. Jr., Litman, B., Kim, H. Y. & Gawrisch, K. (2001) Lipids. 36:945-959; and Gu, X., Meer, S. G., Miyagi, M., Rayborn, M. E., Hollyfield, J. G., Crabb, J. W., and Salomon, R. G. (2003) J. Biol. Chem. 278: 42027-42035. DHA is also envisioned as a target of oxidative stress, mainly by reactive oxygen intermediates that in turn trigger RPE and photoreceptor cell damage.

Rhodopsin mutations in retinitis pigmentosa expressed in rats are associated with a decreased content of DHA in photoreceptors. See, Anderson, R. E, Maude, M. B., McClellan, M., Matthes, M. T., Yasumura, D. & LaVail, M. M. (2002) Mol. Vis. 8:351-358. This observation is interpreted as a retinal response to a metabolic stress, whereby decreasing the amount of the major target of lipid peroxidation, DHA, elicits protection. Retinal degeneration induced by constant light promotes DHA loss from photoreceptors, but rats reared in bright cyclic light are protected. See, Li, F., Cao, W. & Anderson, R, E. (2001) Exp. Eye Res. 73:569-577. These studies suggest a remarkable adaptation/plasticity that may involve endogenous molecules that have not been characterized.

RPE cells also perform several other functions, including transport and reisomerization of bleached visual pigments, and contribute to the maintenance of the integrity of the blood-outer retinal barrier. Retinal detachment or trauma triggers dysfunctions in the RPE cells that lead to the onset and development of proliferative vitreoretinopathy.

RPE cells are essential for photoreceptor cell survival. When RPE cells are damaged or die, photoreceptor function is impaired, and the cells die as a consequence. Thus, oxidative stress-mediated injury and cell death in RPE cells impair vision, particularly when the RPE cells of the macula are affected. The macula is responsible for visual acuity. The pathophysiology of many retinal degenerations (e.g., age-related macular degenerations and Stargardt's disease) involves oxidative stress leading to apoptosis of RPE cells. In fact, RPE cell damage and apoptosis seem to be dominant factors in age-related macular degeneration. See, Hinton, D. R., He, S. & Lopez, P. F. (1998) Arch. Ophthalmol. 116:203-209. In Stargardt's disease, the lipofuscin fluorophore A2E mediates RPE damage. The caspase-3 enzyme has been shown to be part of this cascade, whereas the anti-apoptotic Bcl-2 protein exerts cellular protection. See, Sparrow, J. R. Vollmer-Snarr, H. R., Zhou, J., Jang, Y. P., Jockusch, S., Itagaki, Y. & Nakanishi, K. (2003) J. Biol. Chem. 278:18207-18213.

Oxidative stress triggers multiple signaling pathways. Some are cytoprotective, and others lead to cell damage and eventually cell death. Among these signals are the Bcl-2 family proteins. In fact, expression of pro- and anti-apoptotic Bcl-2 family proteins is altered by oxidative stress and represents a major factor, insofar as the outcome of the apoptotic signaling, since cell survival reflects the predominance of one set of proteins over the other. In the RPE and photoreceptor cells, oxidative stress, induced by several factors including retinal light exposure or reactive oxygen species, triggered an unfavorable shift in the Bcl-2 family proteins toward cell damage. See, Osborne, N. N., Cazevieillem C., Pergandem G. & Wood, J. P. (1997) Invest. Ophthalmol. Vis. Sci. 38:1390-1400; and Liang, Y. G., Jorgensen, A. G., Kaestel, C. G., Wiencke, A. K., Lui, G. M., la Cour, M. H., Ropke, C. H. & Nissen, M. H.(2000) Curr. Eye Res. 20:25-34.

Retinal DHA is a target of oxidative stress-mediated lipid peroxidation. See, Organisciak, D. T., Darrow, R. M., Jiang, Y. L. & Blanks, J. C. (1996) Invest. Ophthalmol. Vis. Sci. 37:2243-2257. Oxidative stress in brain generates neuroprostanes from DHA through an enzyme-independent reaction in brain, and in the retina DHA is thought to be an active site of lipid peroxidation. See, Roberts, L. J. 2nd, Montine, T. J., Markesbery, W. R., Tapper, A. R., Hardy, P., Chemtob, S., Dettbarn, W. D. & Morrow, J. D. (1998) J. Biol. Chem. 273:13605-13612. There are also studies that demonstrate DHA-mediated neuroprotection in photoreceptor cells and brain. See, Rotstein, N. P., Aveldano, M. I, Barrantes, F. J., Roccamo, A. M. & Politi, L. E. (1997) J. Neurochem. 69:504-513; and Rotstein, N. P. Politim, L. E., Germanm, O. L. & Girotti, R. (2003) Invest. Ophthalmol. Vis. Sci. 44:2252-2259; and Kim, H. Y., Akbar, M., Lau, A. & Edsall, L. (2000) J. Biol. Chem. 275:35215-352. To date, no specific DHA mediators have been identified that elicit protection in RPE cells, except certain docosanoids identified in retina, the formation of which is inhibited by lipoxygenase inhibitors. Although the bioactivity of these docosanoids has not been studied, they were suggested to be neuroprotective. See, Bazan, N. G., Birkle, D. L. & Reddy, T. S. (1984) Biochem. Biophys. Res. Commun. 125:741-747; Bazan, N. G. (1989) Prog. Clin. Biol. Res. 312: 95-112; and Bazan, N. G. (2003) J Lipid Res. 44: 2221-2233.

### DISCLOSURE OF INVENTION

We have discovered that the unique DHA product, 10, 17S-docosatriene (“Neuroprotectin D1” or “NPD1”), provides surprisingly effective neuroprotection when administered right after an experimental stroke. Moreover, both nerve cells and retinal pigment epithelial (RPE) cells were found to synthesize 10,17S-docosatriene (NPD1) from DHA. NPD1 also potently counteracted H₂O₂/TNFα oxidative stress-mediated cell apoptotic damage. Under the same oxidative-stress conditions, NPD1 up-regulated the anti-apoptotic Bcl-2 proteins, Bcl-2 and Bcl-xL, and decreased expression of the pro-apoptotic proteins, Bad and Bax. Moreover, in RPE cells NPD1 inhibited oxidative stress-induced caspase-3 activation, IL-1β-stimulated human COX-2 promoter expression, and apoptosis due to N-retinylidene-N-retinylethanolamine (A2E), a cytotoxic compound that accumulates in age-related macular degeneration. Overall, NPD1 protected both nerve and retinal pigment epithelial cells from cellular apoptosis and damage due to oxidative stress. NPD1 concentration was found to be significantly decreased in the hippocampus of Alzheimer's patient brains. In cultured human brain cells, NPD1 synthesis was up-regulated by neuroprotective soluble p amyloid, and NPD1 was found to inhibit secretion of toxic β amyloid peptides.

### BRIEF DESCRIPTION OF DRAWINGS

FIG. 1A illustrates the time course of accumulation of three docosanoid derivatives (17S-HDHA, 10,17S-docosatriene (NPD1), and ω-22-hydroxy-4,17S-diHDHA) in 24 h of reperfusion following 1 h of middle cerebral artery occlusion (MCA-O).

FIG. 1B illustrates a selected ion monitoring (SIM) chromatogram (m/z 359) for 10,17S-docosatriene (NPD1).

FIG. 1C illustrates the chemical structure and MS-MS spectrum for 10,17S-docosatriene (NPD1).

FIG. 1D illustrates the selected ion monitoring (SIM) chromatogram (m/z 375) for ω-22-hydroxy-4,17S-diHDHA.

FIG. 1E illustrates the time course of accumulation of the 17R-series resolvins in the hippocampus of mice pretreated with aspirin in 24 h of reperfusion following 1 h of middle cerebral artery occlusion (MCA-O).

FIG. 1F illustrates a selected ion monitoring (SIM) chromatogram (m/z 375) for 7,8,17R-triHDHA.

FIG. 1G illustrates a selected ion monitoring (SIM) chromatogram (m/z 359), marking the peak for 7,17R-diHDHA marked.

FIG. 1H illustrates a selected ion monitoring (SIM) chromatogram (m/z 327) for docosahexaenoic acid (DHA).

FIG. 2A illustrates the time course of prostaglandin E₂ (PGE₂), leukotriene B₄ (LTB₄), and lipoxin A₄ (LXA4) formation in mouse hippocampus in 24 h of reperfusion following 1 h of middle cerebral artery occlusion (MCA-O).

FIG. 2B illustrates the time course of prostaglandin E₂ (PGE₂), leukotriene B₄ (LTB₄), and lipoxin A₄ (LXA4) formation in hippocampus of mice pretreated with aspirin (7.52 mg/kg; 15 min prior to MCA-O) in 24 h of reperfusion following 1 h of middle cerebral artery occlusion (MCA-O).

FIG. 3 illustrates a proposed biosynthetic pathway for 10,17S-docosatriene (NPD1) and the aspirin-triggered 17R-series resolving.

FIG. 4 illustrates the inhibition of leukocyte infiltration in zymosan-induced peritonitis by 4,17S-diHDHA and 10,17S-docosatriene (NPD1) at two concentrations.

FIG. 5A illustrates the inhibition of leukocyte infiltration as measured by myeloperoxidase activity in the hippocampus and neocortex (both ipsilateral and contralateral sides of MCA-O) of mice following 1 h MCA-O and 48 h of reperfusion, both with and without 0.4 μg 10,17S-docosatriene (NPD1). The small letters refer to the level of statistical significance: a, d, and e indicate p values that are <0.01.

FIG. 5B illustrates the inhibition of leukocyte infiltration as measured by myeloperoxidase activity in the hippocampus and neocortex (both ipsilateral and contralateral sides of MCA-O) of mice following 1 h MCA-O and 48 h of reperfusion, both with and without 1 μg or 3 μg 10,17S-docosatriene methyl ester. The small letters refer to the level of statistical significance: a, d, e and f indicate p values that are<0.03.

FIG. 5C illustrates the inhibition of leukocyte infiltration as measured by myeloperoxidase activity in the hippocampus and neocortex (both ipsilateral and contralateral sides of MCA-O) of mice following 1 h MCA-O and 48 h of reperfusion, both with and without docosahexaenoic acid (DHA) at concentrations of 2 μg, 20 μg, and 200 μg. The small letters refer to the level of statistical significance: a, b, c, e, and f indicate p values that are <0.03; g indicates no significant difference.

FIG. 6 illustrates the percentage of the infarcted volume (stroke volume) with respect to the total brain coronal area in a mouse brain (the damaged area measured by the inability to metabolize triphenyltetrazolium chloride (TTC)) following 1 h MCA-O and 48 h reperfusion, with and without infusion of 1 μg 10,17S-docosatriene (NPD1) during reperfusion.

FIG. 7A illustrates the change in nuclear factor kappa B (NFκB) in mouse hippocampus after 1 h MCA-O followed by 2 h reperfusion, with infusion of either vehicle, 10,17S-docosatriene (NPD1; 0.16 μg/ml), or docosahexaenoic acid (DHA).

FIG. 7B illustrates the change in HIF-1α, AP1, and STAT-1 in mouse hippocampus after 1 h MCA-O followed by 2 h reperfusion, with infusion of either vehicle, 10,17S-docosatriene (NPD1; 0.16 μg/ml), or docosahexaenoic acid (DHA).

FIG. 7C illustrates the change in COX-1 and COX-2 in mouse hippocampus after 1 h MCA-O followed by 2 h reperfusion, with infusion of either vehicle, 10,17S-docosatriene (NPD1; 0.16 μg/ml), or docosahexaenoic acid (DHA).

FIG. 7D illustrates the change in HIF-1 and NFκB concentrations as measured by EMSA in cultured neural progenitor cells exposed to IL-1β, with and without docosahexaenoic acid (DHA) or 10,17S-docosatriene (NPD1) at various concentrations.

FIG. 7E illustrates the change in AP1 and STAT-1 concentrations as measured by EMSA in cultured neural progenitor cells exposed to IL-1β, with and without docosahexaenoic acid (DHA) or 10,17S-docosatriene (NPD1).

FIG. 7F illustrates the change in COX-1 and COX-2 concentrations as measured by EMSA in cultured neural progenitor cells exposed to IL-1β, with and without 10,17S-docosatriene (NPD1).

FIG. 7G illustrates the change in COX-1 and COX-2 mRNA expression in cultured neural progenitor cells exposed to IL-1β, with and without 10,17S-docosatriene (NPD1).

FIG. 8A illustrates the change in Aβ40 peptide concentration in aging human neural cells cultured for 8 weeks (Control, Con), and exposed to either the cytokine IL-1β or docosahexaenoic acid (DHA).

FIG. 8B illustrates the change in Aβ42 peptide concentration in aging human neural cells cultured for 8 weeks (Control, Con), and exposed to either the cytokine IL-1β or docosahexaenoic acid (DHA).

FIG. 8C illustrates the change in 10,17S-docosatriene (NPD1) concentration in aging human neural cells incubated for 8 wk in the presence of Aβ protein (25 μM), sAPPα (20 μM or 100 μM), or DHA (50 nM).

FIG. 8D illustrates the change in free docosahexaenoic acid (DHA) concentration in aging human neural cells incubated for 8 wk in the presence of Aβ protein (25 μM), sAPPα (20 μM or 100 μM), or DHA (50 nM).

FIG. 9 illustrates the difference in 10,17S-docosatriene (neuroprotectin D1; NPD1) concentration in brain tissue from the hippocampal CA1 region of Alzheimer's Disease patients and from age-matched, controls.

FIG. 10A illustrates the results of Western blot analysis on the relative concentrations of Bcl-2, Bfl-1(A1), and actin expression in human neural cells either untreated or treated with Aβ protein, 10,17S-docosatriene (NPD1; 50 nM) or docosahexaenoic acid (DHA; 50 nM).

FIG. 10B illustrates the results of Western blot analysis on the relative signal strength of Bcl-2 and Bfl-1(A1), as normalized with expressed actin, in human neural cells either untreated or treated with Aβ protein, 10,17S-docosatriene (NPD1; 50 nM) or docosahexaenoic acid (DHA; 50 nM).

FIG. 11 A illustrates the structure and the MS-MS spectrum for 10,17S-docosatriene (NPD1), showing a full scan of negative ion products for selected parent ion (m/z 359). The insert is the UV spectrum for 10,17S-docosatriene (NPD1).

FIG. 11B illustrates the change in concentration of free DHA and 10,17S-docosatriene (NPD1) in RPE cells exposed to IL-1β.

FIG. 11C illustrates the change in concentration of free DHA and NPD1 (10,17S-docosatriene) in RPE cells as a function of incubation time with the calcium ionophore A-23187.

FIG. 11D illustrates a possible biosynthetic pathway for NPD1 (10,17S-docosatriene).

FIG. 12A illustrates the change in concentration of NPD1 (10,17S-docosatriene) in human retinal pigment epithelial cells upon the addition of docosahexaenoic acid (DHA).

FIG. 12B illustrates the change in concentration of free DHA and NPD1 (10,17S-docosatriene) in serum-starved retinal pigment epithelial cells as a function of incubation time with free DHA (50 μM) added to the culture.

FIG. 13A illustrates the effect of addition of NPD1 (10,17S-docosatriene; 50 nM) on DNA fragmentation as measured by differential sedimentation after ³H-thymidine labeling in RPE cells treated with TNFα/H₂O₂ to promote oxidative stress.

FIG. 13B illustrates the effect of addition of NPD1 (10,17S-docosatriene; 50 nM) on DNA fragmentation in RPE cells as measured by ELISA detection of mono- and oligonucleosomes in RPE cells treated with TNFα/H₂O₂ to promote oxidative stress.

FIG. 14A illustrates the effect of the addition of NPD1 (10,17S-docosatriene; 50 nM) on the concentration of the anti-apopotic protein Bcl-2 in RPE cells exposed to TNFα and H₂O₂ (400 and 800 μM) to promote oxidative stress.

FIG. 14B illustrates the effect of the addition of NPD1 (10,17S-docosatriene; 50 nM) on the concentration of the anti-apopotic protein Bcl-xL in RPE cells exposed to TNFα and H₂O₂ (400 and 800 μM) to promote oxidative stress.

FIG. 14C illustrates the effect of the addition of NPD1 (10,17S-docosatriene; 50 nM) on the concentration of the pro-apoptotic protein Bad in RPE cells exposed to TNFα and H₂O₂ (400 and 800 μM) to promote oxidative stress.

FIG. 14D illustrates the effect of the addition of NPD1 (10,17S-docosatriene; 50 nM) on the concentration of the pro-apoptotic protein Bax in RPE cells exposed to TNFα and H₂O₂ (400 and 800 μM) to promote oxidative stress.

FIG. 15A illustrates the effect of NPD1 (10,17S-docosatriene; 50 nM) on caspase-3 cleavage in RPE cells during oxidative stress-induced apoptotic conditions using both 800 and 400 μM H₂O₂ in combination with TNFα (10 ng). Caspase-3 cleavage was detected by Western-blot analysis using PARP as antibody.

FIG. 15B illustrates the effect of NPD1 (10,17S-docosatriene; 50 nM) on caspase-3 cleavage in RPE cells stably transfected with a lentivirus construct containing the DEVD caspase-3 cleavage sequence during oxidative stress-induced apoptotic conditions using both 800 and 400 μM H₂O₂ in combination with TNFα (10 ng).

FIG. 16 illustrates the effect of different concentrations of NPD1 (10,17S-docosatriene) (from 0.05 to 100 nM) on the expression of the COX-2 promoter in RPE cells induced by IL-1β. The RPE cells were transfected with a COX-2 promoter-luciferase construct, and luciferase activity measured with a luminometer after addition of luciferin.

### MODES FOR CARRYING OUT THE INVENTION

Neuroprotection Following Ischemic Stroke

Ischemic stroke triggers lipid peroxidation and neuronal injury. Docosahexaenoic acid, released from membrane phospholipids during brain ischemia, is a major source of lipid peroxides. Leukocyte infiltration and pro-inflammatory gene expression also contribute to stroke damage. Using lipidomic analysis, stereospecific messengers from docosahexaenoate-oxygenation pathways were identified in a mouse stroke model. Aspirin, widely used to prevent cerebrovascular disease, activated an additional pathway, which included the 17R-resolvins. As shown below, the newly discovered brain messenger 10,17S-docosatriene (NPD1), potently inhibited leukocyte infiltration, nuclear factor kappa B and cyclooxygenase-2 induction in experimental stroke, and elicited neuroprotection.

### EXAMPLE 1

### Materials and Methods for NPD1 Neuroprotection Following Stroke Damage

Reagents. Human recombinant IL-1β, (14019) was from Sigma Chemical Co. (St. Louis, Mo.), and 10,17-diHDHA was prepared as previously described in Serhan, C. N., Clish, C. B., Brannon, J., Colgan, S., Chiang, N., and Gronert, K. (2000) J. Exp. Med., 192, 1197-1204; and Bederson, J. B., Pitts, L. H., Tsuji, M., Nishimura, M. C., Davis, R. L., and Bartkowski, H. (1986) Stroke 17, 472-476. Normal human neural (HN) progenitor cells (CC-2599), NPMM (neural progenitor maintenance medium), human epidermal and fibroblast growth factors (hEGF, hFGF), gentamicin/amphotericin B (G/A1000), and neural survival factor-1 (NSF-1) were obtained from Clonetics (Walkersville, Md.). AP1, HIF-1α, NF-κBp50/p65, and STAT-1α gel-shift consensus and mutant oligonucleotides were synthesized at the LSUHSC core facility or were purchased from Promega Life Science (Madison, Wis.).

Middle Cerebral Artery Occlusion (MCA-O) and reperfusion. Experimental protocols were reviewed and approved by the Institutional Animal Care and Use Committee of Louisiana State University Health Sciences Center, New Orleans. Mice (20-25 g body wt) were induced with 2% isoflurane in a mixture of 70% nitrous oxide and 30% oxygen. Anesthesia was maintained with 1% isoflurane. Temperature was maintained at 36.5-37.5° C. using a Harvard homeothermic blanket. P 10 polyethylene catheters were placed in the femoral artery and vein, and the blood pressure was monitored. Arterial blood was analyzed for PO₂, PCO₂, and pH after 1 h of ischemia and 30 min after the onset of reperfusion. The common carotid and external carotid arteries were temporarily ligated with a retracting suture, and the external carotid artery was dissected just proximal to its bifurcation. The occluding filament was introduced from the external carotid artery and advanced to the internal carotid artery. The AVM micro clip was removed. The filament was advanced so that the blunted tip lay in the anterior cerebral artery and the side of the filament occluded the origin of the middle cerebral artery (MCA). The stump of the external carotid artery was ligated, and tension on the retracting suture to the common carotid artery was gently released, restoring blood flow to the carotid system.

Animals were allowed to recover from anesthesia, and 15 min before the start of reperfusion, a neurologic performance test was applied as described in Bederson, J. B., Pitts, L. H., Tsuji, M., Nishimura, M. C., Davis, R. L., and Bartkowski, H. (1986) Stroke 17, 472-476. This neurologic test allowed an assessment of the degree of damage. A rating of Class II or Class IV indicated very severe damage; Class I indicated very mild damage. To enhance consistency in the group of selected animals, only mice rated as having a Class II neurologic performance were included in the study. The variables minimized by this screening are anatomic variants of brain arteries and surgical procedure. About 85% of the mice had Class II neurologic performance.

Occlusion of the middle cerebral artery (MCA-O) was maintained for 1 h; reperfusion was then established by delicate retrieval of the occluding suture from the arterial lumen, restoring blood flow to the region of the MCA. The mice were then killed, and the hippocampus and brain cortex rapidly dissected and frozen. Samples were kept at −80° C. until analysis. In some experiments, aspirin (7.5 mg/kg) was administered by gavage 15 min before MCA-O.

Assessment of stroke volume. Forty-eight hours after MCA-O, mice were killed, and brains were dissected and immersed in ice-cold saline. The dissected brains were embedded in agar blocks and sectioned into coronal slices 1 mm thick by a vibratome (Vibratome Co. St. Louis, Mo.). Sections were incubated at room temperature in a 3% buffered solution of 2,3,5,-triphenyltetrazolium chloride (TTC). Once color developed (10 to 15 min), sections were fixed in 10% buffered formalin, and kept at 4° C. until images were recorded by a camera (Cool-snap, Nikon) mounted to a dissecting microscope. In the digital images, total brain area and stroke areas were analyzed and calculated by Adobe Photoshop software. Serial sections were made for all animals. (Digital images not included).

Human neural (HN) progenitor cells in primary culture. HN cells were grown to ˜70% confluence (˜50,000 cells per 3.5-cm diameter well) in 6-well COSTAR plates at 37° C., 5% CO₂/20% O₂/75% N₂ in humidified air at 1 atm (normoxic conditions) in NPMM medium (Clonetics CC-4241) supplemented with hFGF, NSF-1, hEGF, GA-1000, as described by the manufacturer (Clonetics, Walkersville, Md.). HN cells tested negative for HIV-1, hepatitis B and C, mycoplasma, bacteria, yeast, and fungi, and positive for the glial and neuronal markers GFAP, MAP2, and β-tubulin III (Clonetics). After two weeks of development, HN cells were exposed to human recombinant interleukin 1-beta (IL-1β) (10 ng/ml) for 3 h in the presence or absence of 0.03, 0.3, 3.0, 30, and 300 nM 10,17S-docosatriene (NPD1), DHA, or PBS pH 7.4 (control). RNA and protein were quickly isolated using Trizol Reagent (Invitrogen, Carlsbad, Calif.) and stored at −81° C. within minutes of isolation.

COX-2 RNA abundance in hippocampus and HN cells. Abundance of human-specific COX-1 and COX-2 RNA message was assayed using RT-PCR as described in Bazan, N. G., and Lukiw, W. J. (2002) J. Biol. Chem. 277, 30359-30367.

Electrophoretic mobility-shift assay (EMSA) of human AP1, HIF-1α, NF-κBp50/p65, and STAT-1α. Nuclear protein extracts (NPXTs) were prepared from one to three 3.5 cm-diameter wells of HN cells and quantitated. NPXTs (5 μg) derived from HN cells were incubated with[γ-³²P]-ATP (˜3000 Ci/mmol)-end-labeled AP1, HIF-1α, NF-κκBp50/p65, or STAT-1α consensus and mutant oligonucleotides in 5-μl volumes, reacted for 30 min on ice, analyzed on 5% or 10% acrylamide/90 mM Tris-borate pH 8.4, 1 mM EDTA (TBE) gels, dried onto 2-mm Whatman filter paper at 80° C. for 2 h, and phosphorimaged using a Typhoon Variable Mode Imager (Amersham Pharmacia Biotech, Piscataway, N.J. ).

Polymorphonuclear leukocyte infiltration measurement by the myeloperoxidase (MPO) assay. Inhibition of leukocyte infiltration by 10,17S-docosatriene (NPD1) or docosahexaenoic acid (DHA) was measured in mouse hippocampus and neocortex after 1 h of middle cerebral artery occlusion (MCA-O) and 48 h of reperfusion. Lipids were delivered for each experiment by perfusion (250 nl/h) through Alzet minipumps implanted into the third ventricle. Hippocampus and neocortex from the ipsilateral (MCA-O-reperfusion) and contralateral sides were rapidly dissected. Brain samples were assayed for myeloperoxidase activity as described in Huang, J., Choudhri, T. F., Winfree, C. J., McTaggart, R. A., Kiss, S., Mocco, J., Kim, L. J., Protopsaltis, T. S., Zhang, Y., Pinsky, D. J., and Connolly Jr., E. S. (2000) Stroke 31, 3047-3053. Briefly, brain tissues were homogenized in 10 mM phosphate buffer (pH 7.4), then frozen and thawed with liquid N₂ followed by sonication. Samples were precipitated at 10,000×g for 10 min, then aliquots of supernatants were added to 10 mM phosphate buffer (pH 6.0) and a substrate solution containing o-dianisidine (Sigma; St. Louis, Mo.) and 0.025% hydrogen peroxide, and finally incubated at 37° C. for 45 min. Spectrophotometric detection was obtained at 460 nm.

Immunohistochemistry of myeloperoxidase to assess leukocyte infiltration. After MCA-O, mice were infused by Alzet mini-pumps with vehicle or 10,17S-docosatriene (NPD1). After 48 h, mice were anesthetized and killed by intracardial perfusion of ice-cold saline followed by 10% neutral buffered formalin. Brain tissues were allowed to equilibrate overnight in 4% buffered formalin, followed by 30% sucrose in 0.1 M PBS. Frozen sections were made at 10 μm thickness and mounted on glass slides. Sections were permeablized with 0.6% Triton X-100 for 10 min, washed in PBS, and blocked in 2% goat serum in PBS for 30 min. Incubation with myeloperoxidase/FITC-conjugated antibody (DAKO A/S, Denmark) was performed at 1:200 dilution for 2 h. Sections were washed in Tween-20 in PBS then mounted in Vectashield (Vector, Calif.). Images were recorded by deconvolution microscope (Intelligent Imaging Innovations, Denver, Colo.).

LC-MS-MS Analysis of Docosanoids. Quantitative analysis of docosanoids by LC-MS-MS was performed in hippocampus from mice (C57/BL-6, 20-25 g body weight) killed by head-focused microwave radiation at different time points after the onset of reperfusion. The hippocampus was rapidly dissected (20-70 mg wet tissue weight), homogenized in cold methanol, and kept under nitrogen at −80° C. until purification. Purification was performed by SPE technique as described in Hong, S., Gronert, K., Devchand, P. R., Moussignac, R. L., and Serhan, C. N. (2003) J. Biol. Chem. 278, 14677-14687; and Serhan, C. N., Clish, C. B., Brannon, J., Colgan, S., Chiang, N., and Gronert, K. (2000) J. Exp. Med., 192, 1197-1204. In short, samples after pre-equilibration at pH 3.0 were loaded onto C18 columns (Varian), and eluted with 10 ml 1% methanol in ethyl acetate (EM Science). Eluted samples were concentrated by nitrogen-stream evaporator before LC-MS analysis. Concentrated samples were loaded into a Surveyor MS pump (Thermo-Finnegan) equipped with a C18 discovery column (Supelco), 10 cm×2.1 mm ID, 5 μm internal phase. Samples were eluted in a linear gradient[100% solution A (60:40:0.01 methanol/water/acetic acid) to 100% solution B (99.99:0.01 methanol/acetic acid)] and run at a flow rate of 300 μl/min for 45 min. LC effluents were diverted to an electro-spray-ionization probe (ESI) on a TSQ Quantum (Thermo-Finnegan) triple quadrupole mass spectrometer running on negative ion-detection mode. Docosanoid standards were used for calibration and optimization. The instrument was run on full-scan mode, to detect parent ions, and on selected-reaction mode (SRM) for quantitative analysis to detect daughter ions simultaneously. The selected parent ions were 327 for DHA and 359 for 10,17S-docosatriene (NPD1); and the daughter ions were 325.1 and 297, respectively.

### EXAMPLE 2

### Brain Ischemia-Reperfusion Triggered the Synthesis of Docosahexaenoic Acid-Oxygenation Pathways

Right middle cerebral artery occlusion in mice for 1 h followed by reperfusion was used to assess the formation of docosahexaenoic acid (DHA)-oxygenation derivatives. Under these conditions there is active release of free docosahexaenoic acid from brain membrane phospholipids. This model of transient focal ischemia greatly affects the hippocampus, a brain region rich in neurons vulnerable in ischemic stroke, and in other neurologic diseases as described in Bazan, N. G., and Allan, G. (1998) In Cerebrovascular Disease: Pathophysiology, Diagnosis, and Management, eds. Ginsberg, M. D. and Bogousslavsky, J. (Blackwell Science, Inc., Maiden, Mass.) pp. 532-555.

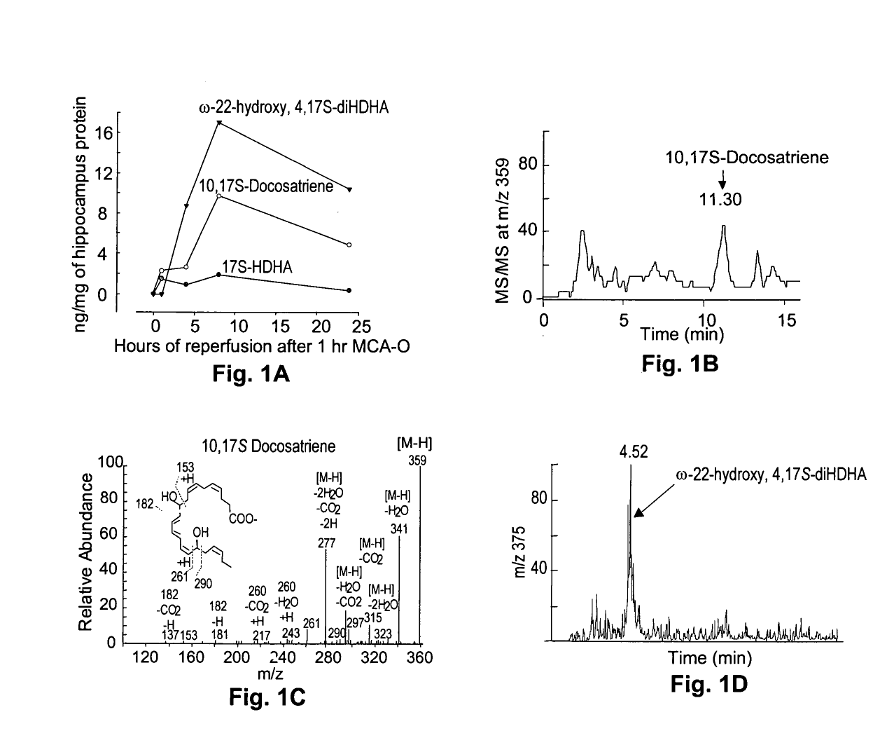

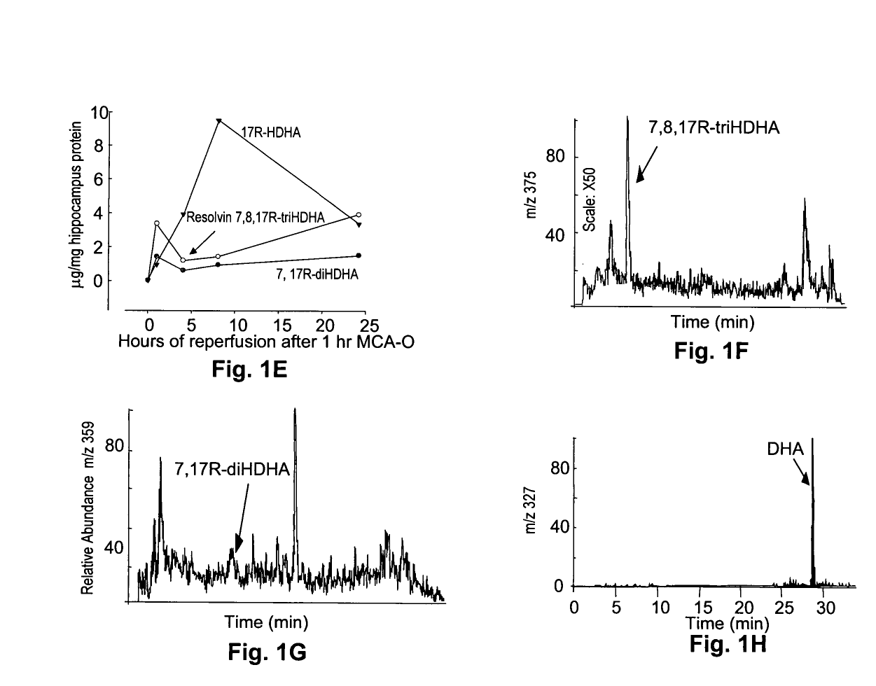

FIGS. 1A-1H indicate the synthesis and metabolism of docosanoids in the ipsilateral mouse hippocampus during reperfusion following transient ischemia. The structural elucidation of DHA and docosanoids was performed by lipidomic analysis by LC-PDA-MS-MS as previously described above in Example 1. FIG. 1A indicates the time course of accumulation of 17S-HDHA, 10,17S-docosatriene (NPD1), and ω-22-hydroxy-4,17S-diHDHA in the ipsilateral hippocampus during reperfusion after 1-h MCA-O. There was an enhanced accumulation of ω-22-hydroxy-4,17S-diHDHA, a product of an oxidative pathway, and 10,17S-docosatriene (NPD1).

FIG. 1B illustrates a selected ion monitoring (SIM) chromatogram (m/z 359) showing 10,17S-docosatriene (NPD1), and FIG. 1C shows the MS-MS spectrum for 10,17S-docosatriene (NPD1). The MS-MS spectrum of 10,17S-docosatriene (NPD1) (FIGS. 1B and C) corresponded to a dihydroxy-containing DHA with prominent fragment ions at m/z 359 (M−H; FIG. 1C). There were also prominent fragment ions at m/z 323 (M−H-2H₂O), 315 (M-H—CO₂), 297 (M−H—H₂O—CO₂), and 277 (M−H-2H₂O—CO₂.2H). Other diagnostic ions were essentially identical to those recently documented in murine brain, human blood, and glial cells. (Hong et al., 2003).

In addition, there was a time-dependent formation of the carbon 22-omega hydroxylation product, 4,17di-HDHA (FIG. 1A) as determined by analysis (at m/z 375) of the MS-MS spectrum (FIG. 1D). FIG. 1D illustrates the SIM (m/z 75) chromatogram showing ω-22-hydroxy-4,17S-diHDHA.

Because aspirin is often used prophylactically as well as therapeutically to manage cerebrovascular diseases, we next tested whether brain biosynthesis of DHA messengers is modified in the presence of aspirin in vivo. Moreover, in non-neural tissues, aspirin triggers the biosynthesis of anti-inflammatory lipid mediators. See, Serhan, C. N., Clish, C. B., Brannon, J., Colgan, S., Chiang, N., and Gronert, K. (2000) J. Exp. Med, 192, 1197-1204. The formation of DHA-derived docosanoids in the presence of aspirin during reperfusion after an ischemic stroke was examined.

FIG. 1E indicates the time course for the accumulation of the 17R-series resolvins in hippocampus of mice pretreated with aspirin before MCA-O. Aspirin triggered a metabolic shift towards accumulation of 17R-docosatrienes, 7,8,17R-triHDHA, and 7,17R-diHDHA, products of acetylated COX-2 enzyme. FIG. 1E shows a shift away from the products generated from endogenous sources of DHA in the absence of aspirin (as seen in FIG. 1A) toward products that include the novel 17R-series resolving, in particular 7,17R-diHDHA and 7,8,17R-triHDHA, which were formed and were present at the earliest time intervals (FIG. 1E). There was a marked accumulation in 17R-HDHA, shown recently to be a product of aspirin-acetylated cyclooxygenase-2. See, Serhan, C. N., Hong, S., Gronert, K., Colgan, S. P., Devchand, P. R., Mirick, G., and Moussignac, R. L. (2002) J. Exp. Med., 196, 1025-1037.

FIG. 1F shows a representative SIM (m/z 375) chromatogram with peak for 7,8,17R-triHDHA; and FIG. 1G shows a representative SIM (m/z 359) chromatogram with peak for 7,17R-diHDHA. FIGS. 1F and G show the LC-MS-MS analysis of the ipsilateral hippocampus following treatment with aspirin, and depict the chromatographic profile of products identified with MS-MS and lipidomic analyses. Finally, FIG. 1H shows the typical SIM (m/z 327) chromatogram with peak for DHA, which confirmed the release of DHA under these conditions.

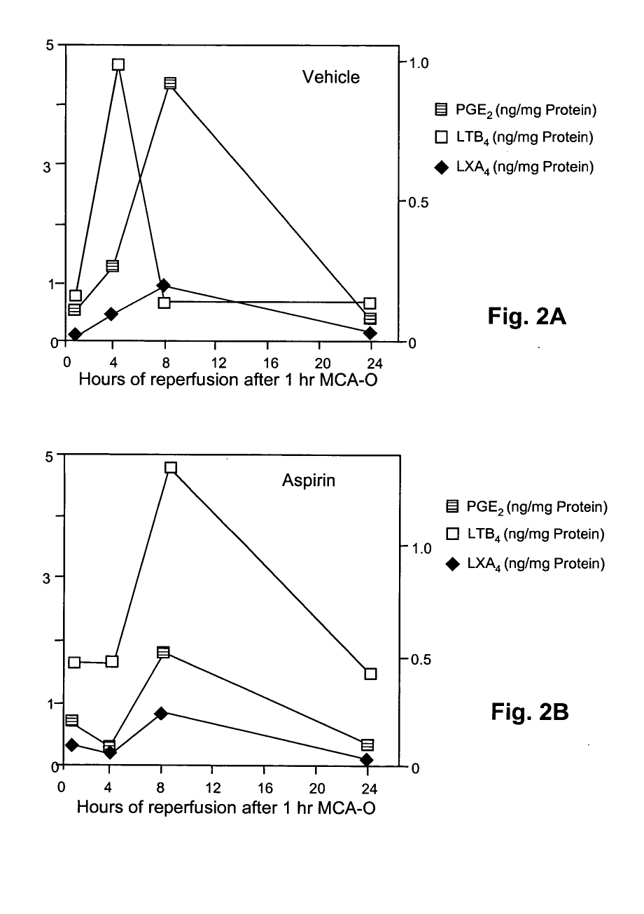

To confirm that the aspirin dose administered by gavage did reach the brain, the time course for prostaglandin E₂, LTB₄, and lipoxin A₄ production was determined in the hippocampus in parallel assays. The time course of prostaglandin E₂, leukotriene B₄, and lipoxin A₄ formation was followed in mouse hippocampus after 1-h middle cerebral artery occlusion (MCA-O), followed by reperfusion with (FIG. 2B) or without (FIG. 2A) aspirin treatment. Detection of the compounds was performed by sensitive and specific ELISA, in tandem, for prostaglandin E₂ (PGE₂), leukotriene B₄ (LTB₄), and lipoxins (LXA₄) (Neogen, Lexington, Ky.).

This study showed inhibitory changes in the eicosanoids when animals were pretreated with aspirin (15 min) before MCA-O. (FIG. 2B) The C57BL/6 mice were treated by gavages with vehicle (sterile saline) or aspirin (7.52 mg/kg). The rapid accumulation of PGE₂ within 8 h of reperfusion as seen in FIG. 2A was inhibited by aspirin treatment as shown in FIG. 2B. Maximal LTB₄ was generated in aspirin-treated hippocampus by 8 h, as was the case with lipoxin A₄, which also peaked within 8 h. Together these results clearly indicate that the dose of aspirin used in vivo did access the central nervous system.

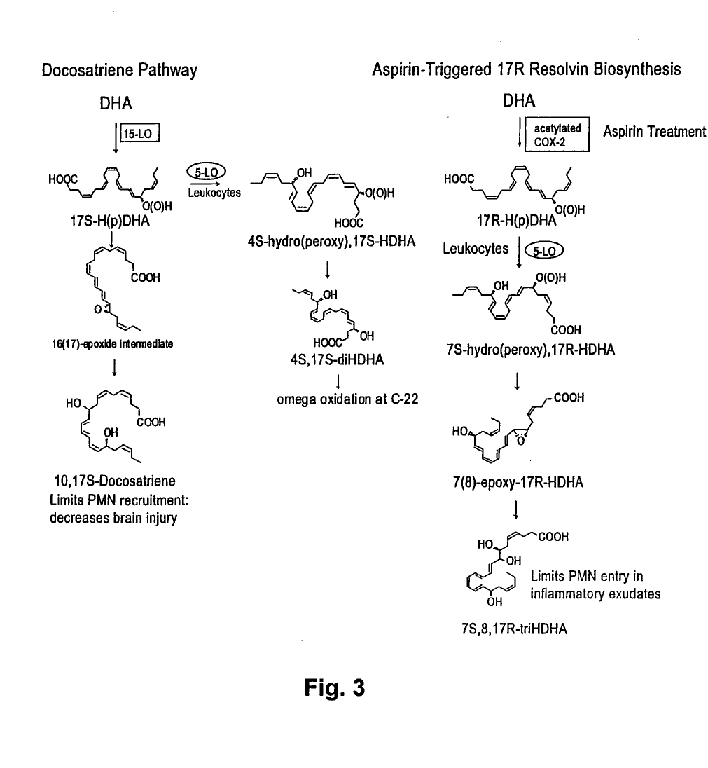

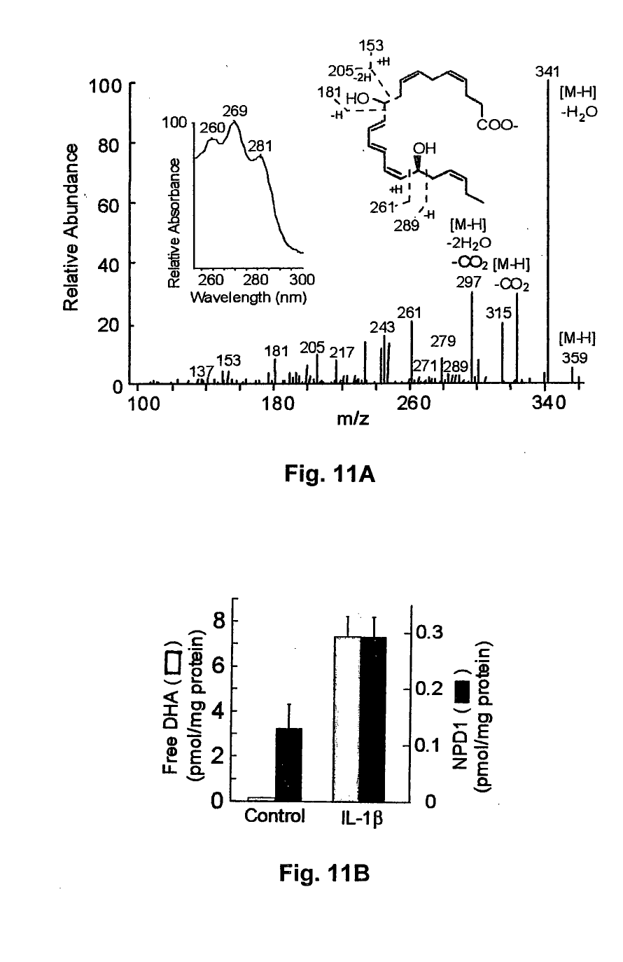

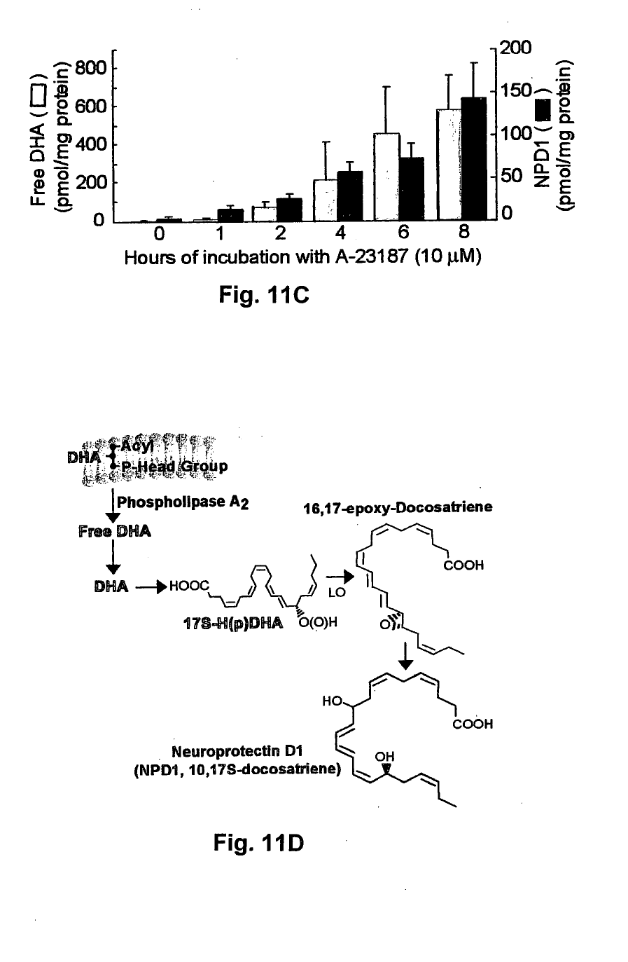

FIG. 3 depicts the proposed biosynthetic pathways for 10,17S-docosatriene (NPD1) and the aspirin-triggered 17R-series resolvins. See, Hong, S., Gronert, K., Devchand, P. R., Moussignac, R. L., and Serhan, C. N. (2003) J. Biol. Chem. 278, 14677-14687; and Serhan, C. N., Clish, C. B., Brannon, J., Colgan, S., Chiang, N., and Gronert, K. (2000) J. Exp. Med., 192, 1197-1204. FIG. 11D further illustrates the steps involved in NPD1 biosynthesis.

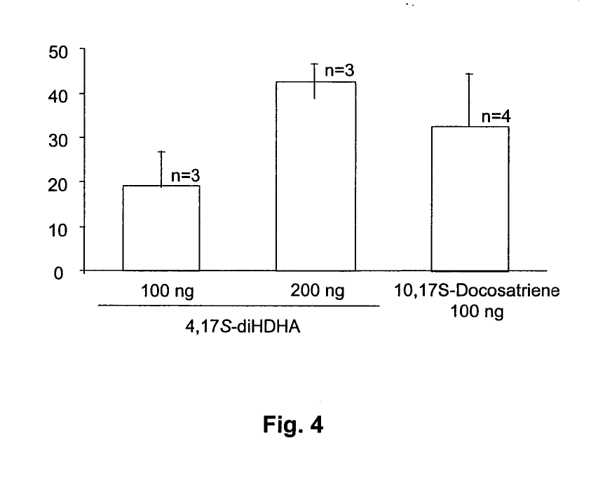

The inhibition of leukocyte infiltration in zymosan-induced peritonitis by 4,17S-diHDHA and 10,17S-docosatriene (NPD1) was tested. The 4,17S-diHDHA caused dose-dependent inhibition of polymorphonuclear leukocyte infiltration. Peritonitis was induced in 6- to 8-week-old male FVB mice (Charles River Laboratories) by peritoneal injection of 1 mg Zymosan A. The compounds 4,17S- and 10,17S-diHDHA were injected by intravenous bolus injection, 1.5 minutes before Zymosan A treatment. Two hours after induction of peritonitis, rapid peritoneal lavages were collected, and cell type enumeration was performed. As shown in FIG. 4, 100 ng 10,17S-docosatriene (NPD1) caused potent inhibition.

In these experiments, 4,17S-diHDHA showed some inhibition of PMN infiltration, but was less than that evoked by equimolar concentrations of 10,17S-docosatriene (NPD1) in a non-neural experimental model (FIG. 4).

### EXAMPLE 3

### Polymorphonuclear Leukocyte Infiltration Mediated by Focal Ischemic Stroke in Mice is Inhibited by 10,17S-docosatriene (NPD1)

Polymorphonuclear leukocyte (PMN) infiltration, a major factor in mediating brain ischemia-reperfusion damage was monitored. PMN infiltration is a complex, multi-step process that is modulated by the coordinated expression of adhesion and signaling molecules. DHA-derived messengers were very recently reported to inhibit PMN invasion outside the central nervous system, in the air-pouch model. (Hong et al., 2003).

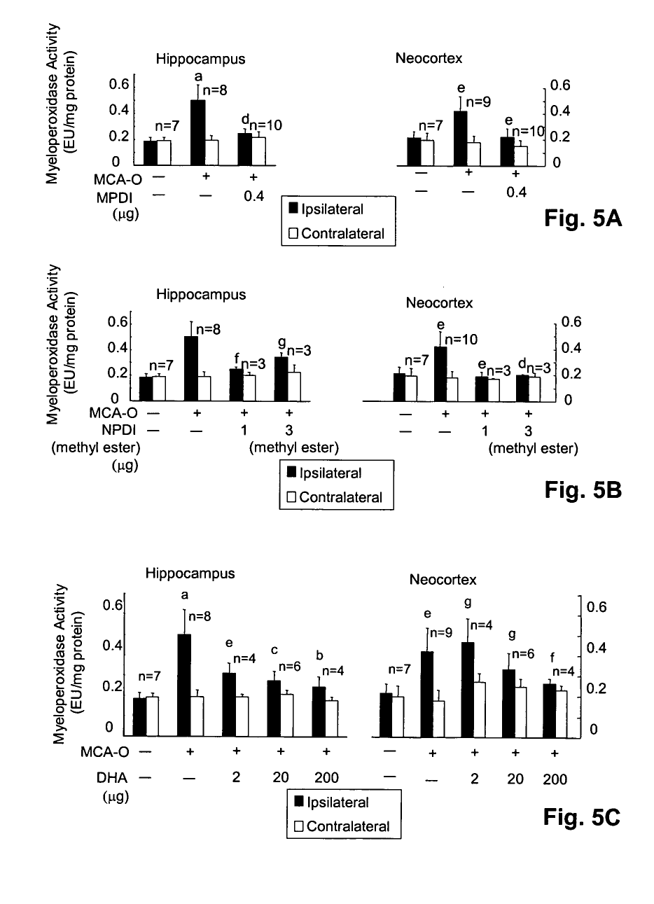

To determine whether 10,17S-docosatriene (NPD1) affected brain ischemia-reperfusion-induced PMN infiltration, either DHA or 10,17S-docosatriene (NPD1) was constantly infused into the third ventricle of a mouse brain during 48 h of reperfusion. FIGS. 5A, 5B, and 5C show the inhibition of leukocyte infiltration by 10,17S-docosatriene (NPD1) in mouse hippocampus and neocortex after 1-h MCA-O and 48 h of reperfusion. The data shown represent averages± SD of the indicated number (n) of individual mice. Immunocytochemical changes in polymorphonuclear leukocytes were shown by a green fluorescence that indicated myeloperoxidase immunoreactivity. (Data not shown). Ipsilateral brain areas (ischemic area) showed positive green fluorescence (vehicle) as compared with contralateral tissue. The fluorescent areas corresponded to the dentate gyrus of the hippocampus. An infusion of 10,17S-docosatriene (NPD1) (0.4 μg over 48 h) exhibited a surprisingly large reduction in green fluorescence. (Photo not shown; Data shown in FIG. 5A).

When viewed with a higher magnification, the ipsilateral stroke area from vehicle-treated animals showed the cytoplasmic granular appearance of immunoreactivity that is characteristic of PMN leukocytes. (Data not shown.)

Cerebroventricular perfusion of 0.4 μg 10,17S-docosatriene (NPD1) over 48 h resulted in 80% inhibition of ipsilateral myeloperoxidase activity as compared to vehicle-treated animals (FIG. 5A). Perfusion with 1 μg or 3 μg 10,17S-docosatriene (NPD1) methyl ester over 48 h resulted in inhibition of ipsilateral myeloperoxidase activity (FIG. 5B). The free acid exerted more potent inhibition (80% and 99% in hippocampus and cortex, respectively) as compared to the methyl-ester derivative, which resulted in 49% inhibition in hippocampus and 97% inhibition in neocortex. (FIGS. 5A and 5B).

The results of perfusion with 2, 20, or 200 μg of DHA (free acid) over 48 h are shown in FIG. 5C. In hippocampus, a dose-dependent inhibition of myeloperoxidase activity was observed (60%, 72%, and 82%, respectively). In neocortex no inhibition was found with 2 μg, but there was 40% and 73% inhibition with 20 or 200 μg, respectively.

In FIGS. 5A, 5B, and 5C, statistically significant differences were determined by the factorial analysis of variance with comparison of treatment means done by protected T-test. The statistical results are indicated by the small letters above the bars: a: P<0.0001; b: P<0.001; c: P<0.003; d: P<0.006; e: P<0.01; f: P<0.03; and g: no significant difference.

Thus, by immunostaining of myeloperoxidase as a marker of PMN leukocytes, a marked inhibition by 10,17S-docosatriene (NPD1) was demonstrated on the ipsilateral side of the brain (FIGS. 5A-5C, photos not shown). Myeloperoxidase is a marker of PMN infiltration in brain, and is depicted with a characteristic cytoplasmic granular pattern found only in the focal ischemic side. (Photo not shown) In addition, hippocampus as well as neocortex was dissected from the focal stroke side (ipsilateral) as well as from the contralateral side, and the myeloperoxidase enzyme activity was quantified. Both the free acid (DHA) as well as the carboxy-methyl ester of 10,17S-docosatriene (NPD1) inhibited the appearance of myeloperoxidase, both in the ipsilateral hippocampus and the ipsilateral neocortex. However, in the contralateral brain regions neither compound indicated an increased enzyme activity nor a change in immunostaining for leukocytes.

Infusion of DHA elicited inhibition of PMN infiltration mainly in the hippocampus (FIG. 5C). Without wishing to be bound by this theory, we believe that exogenously infused DHA may be utilized for the synthesis of the bioactive docosanoid, 10,17S-docosatriene (NPD1). This theory is also supported by the finding that after 1-h middle cerebral artery occlusion (MCA-O) followed by 6 h of reperfusion with DHA, 10,17S-docosatriene (NPD1) was unequivocally generated in hippocampus. (Data not shown) However, the infusion of NPD1 was highly effective at a much lower concentration (0.4 μg) than the comparable effective concentration of DHA (200 μg).

### EXAMPLE 4

### 10,17S-docosatriene (NPD1) Elicits Neuroprotection in Mouse Focal Ischemic Stroke.

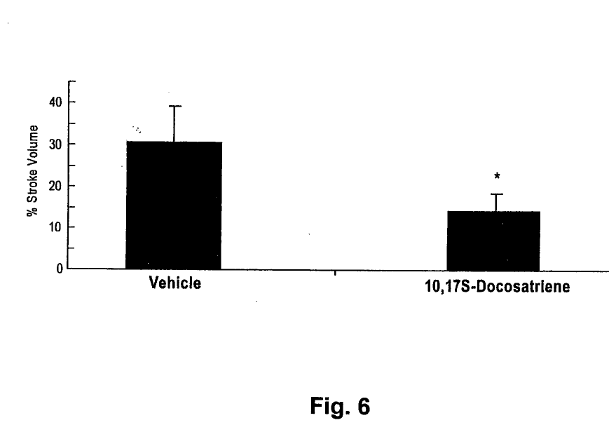

To test for in vivo bioactivity of 10,17S-docosatriene (NPD1) in brain during focal ischemic stroke, Alzet mini-pumps were implanted into the third ventricle to deliver the lipid messenger over the 48 h reperfusion following 1 h of MCA-O. Brain coronal sections were incubated at room temperature in 3% buffered TTC (triphenyltetrazolium chloride) for approximately 10-15 min. The docosanoid 10,17S-docosatriene (NPD1) was infused (1 μg over 48 h by implanted Alzet mini-pumps). In photos, colorless areas indicated mitochondrial damage reflecting the inability to metabolize TTC. (Photos not shown) FIG. 6 depicts the percentage of the TTC-stained area with respect to total brain coronal area. The bars are averages± SD from 6 animals each, and asterisks indicate P<0.0016, by a Student t test.

The focal infarcted volume in mice infused with vehicle was about 30% of the total volume. When 10,17S-docosatriene (NPD1) was infused, there was a reduction to less than 15% of the stroke volume (FIG. 6). These data show the surprising effectiveness of 10,17S-docosatriene (NPD1) in reducing brain damage due to an ischemic stroke.

### EXAMPLE 5

### The Ischemia-Induced Up-Regulation of COX-2 Expression and NF-κB Activation in Brain is Inhibited by 10,17S-docosatriene (NPD1)

Pro-inflammatory gene expression is one promoter of ischemic brain injury. Nuclear factor kappa B (NFκB) is activated in MCA-O, as is cyclooxygenase (COX)-2 expression. COX-2 generates prostaglandin(PG) H₂, the substrate for prostaglandin synthetase, and a contributor to oxidative stress. To investigate whether pro-inflammatory gene expression is a target of the novel docosanoid messenger 10,17S-docosatriene (NPD1), DNA-binding activity of NF-κB and other transcription factors as well as COX-1 and -2 expression was measured in the hippocampus after MCA-O.

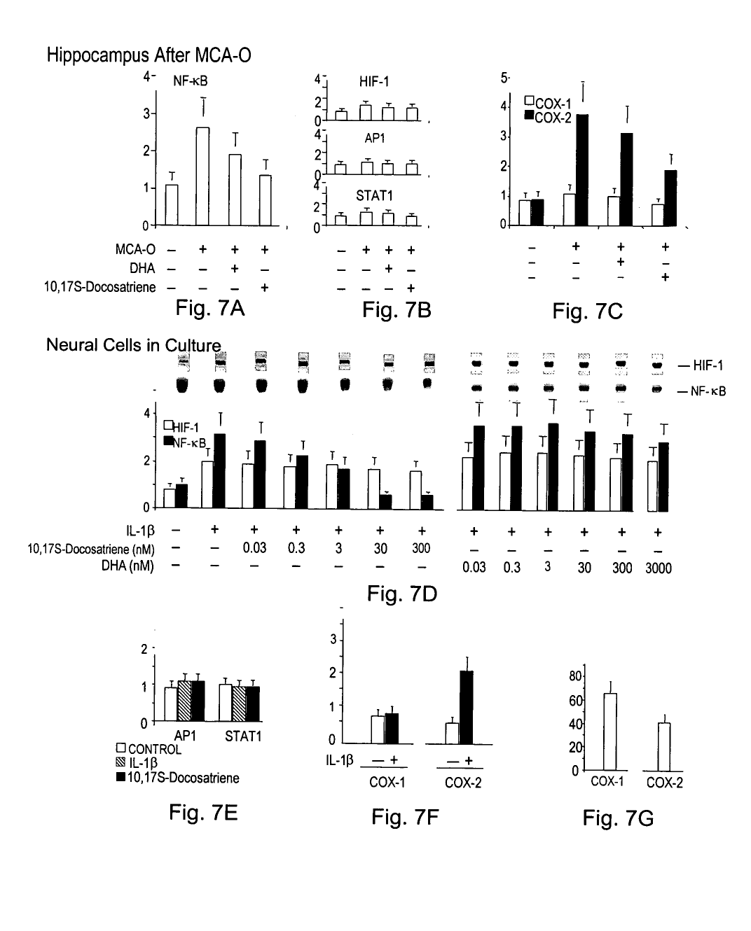

10,17S-docosatriene (NPD1) inhibited MCA-O- and IL-1β-induced NF-κB activation and COX-2 expression as shown in FIGS. 7A-7G. FIGS. 7A, 7B, and 7C show mouse hippocampus after 1 h MCA-O was followed by 2 h reperfusion, where either vehicle, DHA, or 10,17S-docosatriene (NPD1) (0.16 μg/ml) was infused into the third ventricle for 3 h at 0.25 μl/h. FIG. 7A indicates that the enhanced NF-κB-DNA binding activity, determined by EMSA after MCA-O, was inhibited by either 10,17S-docosatriene (NPD1) or DHA. NF-κB was increased over two fold in the ipsilateral hippocampus after MCA-O, and the infusion of either DHA or 10,17S-docosatriene (NPD1) inhibited ischemia-reperfusion-induced NF-κB activation by 28% and 48%, respectively (FIG. 7A).

FIG. 7B shows that AP1 and STAT-1, unlike HIF-1α, were not affected by MCA-O. Only STAT-1-DNA binding was reduced by 10,17S-docosatriene (NPD1). This indicates that the effect was selective, since DNA binding for AP1, HIF1α, and STAT-1, which were slightly enhanced by MCA-O, was unaffected by lipid messengers, except for a small effect of 10-17S-docosatriene (NPD1) on STAT-1-DNA binding (FIG. 7B).

To determine COX-1 and COX-2 expression, mRNA abundance in the ipsilateral hippocampus was measured. FIG. 7C indicates that COX-2 expression was greatly increased by MCA-O-reperfusion in hippocampus, and both 10,17S-docosatriene (NPD1) and DHA inhibited this enhanced expression. A 3-fold increase of COX-2 was seen due to MCA-O reperfusion (FIG. 7C). The infusion of the precursor DHA as well as its product 10,17S-docosatriene (NPD1) inhibited COX-2 expression by 14% and 52%, respectively. The infusion of 10,17S-docosatriene (NPD1) also decreased COX-1 expression by 19%. (FIG. 7C) These results indicate the much greater bioactivity of NPD1 than of DHA.

### EXAMPLE 6

### Selective Inhibition by 10,17S-Docosatriene (NPD1) of IL-1β-Mediated COX-2 Expression and NF-κB Activation in Human Neural Progenitor Cells.

To test whether 10,17S-docosatriene (NPD1) affects pro-inflammatory gene signalling, human neural progenitor cells in culture were used and exposed to the cytokine IL-1β. These cells are known to display dendrites and express neuronal markers. See, Bazan, N. G., and Lukiw, W. J. (2002) J. Biol. Chem. 277, 30359-30367.

IL-1β prominently activated NF-κB, and 10,17S-docosatriene (NPD1) down-regulated NF-κB to a level below that of unstimulated cells in a concentration-dependent manner (FIG. 7D). IL-1β-induced NF-κB activation, but not IL-1β-induced HIF-1α activation, was inhibited by 10-17S-docosatriene (NPD1) as seen in FIG. 7D. Free docosahexaenoic acid (DHA) added in concentrations up to 30 μM was ineffective in modulating either NF-κB or HIF-1α induction by IL-1β. HIF1-α was not affected even at ten-fold higher concentrations of DHA. This is in stark contrast to the inhibitory effect of NPD1 at concentrations as low as 0.3 nM.

As monitored by EMSA and shown in FIG. 7E, 10,17S-docosatriene (NPD1) did not affect the small increase in IL-1β-induced AP1-DNA binding activity; STAT-1 was unaffected by IL-1β or the lipid under these conditions. Although IL-1β slightly increased AP-1-DNA binding activity, 10,17S-docosatriene (NPD1) did not affect the level of this transcription factor or that of STAT-1 under these conditions (FIG. 7E).

The action of 10,17S-docosatriene (NPD1) on intact cells strongly suggests the presence of a receptor-mediated mechanism. Moreover, the lack of effect of DHA implies that, unlike in whole brain, where infused DHA shows some inhibition of PMN infiltration induced by ischemia-reperfusion, in these cultured neural cells exogenous DHA might not be converted to docosanoids in high enough concentrations. Alternatively, other cells may be required for this conversion to occur (e.g., glial cells).

FIGS. 7F and G show that IL1-β-induced expression of COX-2 in neural progenitor cells was decreased by 10,17S-docosatriene (NPD1). IL-1β prominently activated COX-2 expression but not that of COX-1, as shown in FIG. 7F. IL-1β-induced expression of COX-2 mRNA was inhibited by 10,17S-docosatriene (NPD1) (p<0.05), as shown in FIG. 7G.

It is interesting that IL1-β did not activate COX-1 expression, but that NPD1 seemed to decrease the basal level of COX-1 mRNA. Taken together, these observations demonstrated that, after MCA-O, as well as in cytokine-stimulated neural cells in culture, 10,17S-docosatriene (NPD1) produced an overall attenuation of pro-inflammatory gene activation by inhibiting cytokine-induced (and brain ischemia-reperfusion-induced) NF-κB and COX-2 expression.

It has been demonstrated for the first time in a brain undergoing ischemia-reperfusion the generation of stereospecific DHA-oxygenation pathways that lead to the formation of novel neuroprotecting messengers. Two DHA-oxygenation pathways were found: the first pathway is responsible for the formation of the messenger 10,17S-docosatriene (NPD1), and the second pathway, which is active in the presence of aspirin, leads to the formation of the resolvin-type messengers (17R-DHA). The pathways described have the potential for exerting counter-regulatory actions on cellular and molecular signaling that promotes brain injury. In the presence of aspirin and during ischemia-reperfusion, the formation of characteristic DHA messengers were found that in non-neural tissues are potent mediators of inflammation resolution. See, Serhan, C. N., Hong, S., Gronert, K., Colgan, S. P., Devchand, P. R., Mirick, G., and Moussignac, R. L. (2002) J. Exp. Med 196, 1025-1037. These resolvin-type DHA-derived messengers may elicit additional neuroprotective actions in brain ischemia-reperfusion.

The above data demonstrate that the novel 10,17S-docosatriene (NPD1) is a potent inhibitor of ischemia-reperfusion-induced PMN infiltration and pro-inflammatory gene induction. This novel DHA messenger also inhibits cytokine-mediated pro-inflammatory gene activation in neural cells in culture. Overall, 10,17S-docosatriene (NPD1) potently elicited neuroprotection in vivo by reducing the stroke infarct volume 48 h after MCA-O.

In the presence of aspirin, there was enhanced formation in the brain of 17R-series resolvins that have recently been found to be cytoprotective and counter-regulators of inflammation outside the nervous system. See, Serhan, C. N., Clish, C. B., Brannon, J., Colgan, S., Chiang, N., and Gronert, K. (2000) J. Exp. Med, 192, 1197-1204.

The synthesis of 10,17S-docosatriene (NPD1) after 1 h of MCA-O coincided with free DHA availability that results from phospholipase A₂ activation, as well as with reperfusion reoxygenation. The endogenous brain synthesis of 10,17S-docosatriene (NPD1) that peaks at 8 h of reperfusion may be a response of insufficient magnitude to counteract leukocyte infiltration and pro-inflammatory gene induction under the present experimental conditions. Thus the relatively large ischemic insult produced by 1 h of MCA-O followed by several hours of reperfusion (FIG. 6) may overcome the ability of the endogenously generated docosanoids to elicit neuroprotection. Thus, exogenous administration of 10,17S-docosatriene (NPD1) directly into the cerebroventricular system through continuous infusion during the initial two days of reperfusion exerted neuroprotection.

This work has demonstrated that 10,17S-docosatriene (NPD1) continuously administered by intracerebroventricular perfusion inhibited PMN infiltration and other cytokine-induced reactions, and subsequently reduced the stroke volume by 50% after 48 h. The amount of docosanoid infused was 1 μg over a 48-h period, at a rate of 250 nl/h. This observation indicates that 10,17S-docosatriene (NPD1) is a potent neuroprotective compound.

The bioactivity of the novel docosanoid 10,17S-docosatriene (NPD1) was further studied at the cellular level. We chose IL-1β as the trigger, because this cytokine increases during brain ischemia-reperfusion as a result of PMN infiltration as well as activation of microglia and macrophages. See, Mabuchi, T., Kitagawa, K., Ohtsuki, T., Kuwabara, T., Yagita, Y., Yanagihara, T., Hori, M., and Matsumoto, M. (2000) Stroke 31, 1735-1743. Whether the bioactivities of 10,17S-docosatriene (NPD1) in inhibiting PMN infiltration and blocking pro-inflammatory gene expression are independent events or part of the same signalling remains to be defined. What is clear is that the outcome of infusing 10,17S-docosatriene (NPD1) is a large reduction in ischemia-reperfusion neuronal damage.

Protection from Alzheimer's Disease and Nerve Cell Apoptosis

Further experiments were conducted to investigate the synthesis and effects of DHA and neuroprotectin D1 (NPD1), a dihydroxy-containing 10,17S docosatriene (NPD1), in brain cells. As shown below, DHA/NPD1 biosynthesis and signaling was measured in aging, in cytokine- and oxidation-stressed human neural (HN) cells in primary culture, and in Alzheimer's disease (AD) brain. As the accumulation of amyloid beta (Aβ) peptides from beta-amyloid precursor protein (βAPP) is central to AD neuropathology, experiments were conducted to test whether DHA/NPD1 modulates Aβ peptide production during the aging of control and stressed HN cells, and whether sAPPα, a neurotrophic derivative of βAPP, is an inducer of NPD1 biosynthesis, affecting the secretion of neurotoxic Aβ peptides. As shown below, DHA/NPD1 signaling modulated brain neurodegeneration through an up-regulation in the expression of anti-apoptotic members of the Bcl-2 gene family. Taken together, the data below indicate that normal brain cell function and repair are enhanced by DHA and NPD1, and that NPD1 could protect from brain changes seen in AD and in normal aging.

### EXAMPLE 7

### Methods for Experiments Involving Aging Nerve Cells and Tissue from Alzheimer's Disease and Control Brains

Human Neural (HN) Cells. HN cells in primary culture, starting as primary spheroids (CC-2599, Clonetics, Walkersville, Md.), were grown in neural progenitor maintenance medium (NPMM) containing human epidermal and fibroblast growth factor, gentamicin/amphotericin (G/A1000), and neural survival factor-1 for up to 8 weeks. The HN cells showed approximately equal populations of neurons and glia after 3 weeks of development, and stained positive with the neuronal-specific markers NeuN and the glial-specific marker GFAP, as described in Bazan, N. G., Lukiw, W. J. (2002) J. Biol. Chem., 277: 30359-30367. (Data not shown).

Additions of IL-1β and DHA to the Growth Medium. After 1 week of HN cell growth, IL-1β (10 ng/ml) or DHA (50 nM) was added to the NPMM culture medium. NPMM was completely changed every 3 days (with or without IL-1β or DHA), and the concentration of Aβ40 and Aβ42 peptides was assayed every 7 days using Western immunoblot assay as described below. The secretion by HN cells of Aβ42 peptide was approximately one-tenth that of Aβ40 peptide in both control and treated cells (FIGS. 1G and H).

Control and Alzheimer's Disease (AD) Brain Tissues. Brain tissues were used in strict accordance with IRB/ethical guidelines at each donor institution (Louisiana State University Neuroscience Center and the Oregon Health Sciences Center). Samples encompassing the cornu ammonis 1 (CA1) region exhibited no significant differences in age (69.0±1.5 vs 70.3±1.9 yr, p<0.87), post-mortem interval (2.1±0.7 vs 2.0±0.7 h, p<0.96) or tissue pH (6.75±0.1 vs 6.76±0.1, p<0.98), control vs AD, respectively. AD tissues were obtained from patients with a clinical dementia rating (CDR) of 2 or 3 as described by S. S. Mirra et al., (1991) Neurobiology, 41: 478-86. There were no significant differences between the age or post-mortem interval between these two brain groups (p>0.05). There were no significant differences between the RNA yield or RNA spectral quality between these two groups. Total protein was determined using dotMETRIC protein microassay (Chemicon; sensitivity 0.3 ng protein ml⁻¹) using whole brain nucleoprotein as a standard as described in Lukiw, W. J., Pelaez, R. P., Martinez, J., Bazan, N. G. (1998) Proc. Nat. Acad. Sci. (USA), 95: 3914-3919.

Western Analysis. Total protein was extracted, and 25 μg of protein was analyzed by Western blots using human Bcl-2, Bfl-1 (A1) and actin antibodies. Human-specific anti-Bcl-2 [Cat. No. B3170], anti-Bcl-xL[Cat. No. B9429], anti-Bax[Cat. No. B8429] and anti-Bad[Cat. No. B0684] primary antibodies that exhibit no cross-reactivity were obtained from Sigma-RBI (St. Louis, Mo.). Human specific anti-Bfl-1(A1)[Cat. No. sc-8351] was obtained from Santa Cruz Biotechnology (Santa Cruz, Calif.). For Western analysis, signal-intensity data were gathered by phosphorimaging onto molecular imaging screens (FUJIFILM) for 3-36 h using a Typhoon Molecular Imager system (Amersham Biosciences) as described in Bazan and Lukiw, 2002.

Isolation of Lipids from Brain and Cell Lines—LC-MS/MS of DHA and NPD1. Lipids were extracted by homogenization in chloroform/methanol solutions and stored under nitrogen at −80° C. as previously described above in Example 1. For signal quantification, lipid extracts were supplemented with deuterated internal standards, purified by solid-phase extraction, loaded onto a Biobasic-AX column (Thermo-Hipersyl-Keystone; 100 mm×2.1 mm; 5-μm particle sizes), and eluted with a 45-min gradient protocol, starting with solvent solution A (40:60:0:01 methanol:water:acetic acid, pH 4.5; 300 μl min⁻¹). The gradient typically reached 100% solvent B (99.99:0.01 methanol:acetic acid) in 30 min, and was then run isocratically for 5 min. A TSQ Quantum (Thermo-Finnigan) triple quadrupole mass spectrometer and electrospray ionization was used with spray voltage of 3 kV and N₂ sheath gas (35 cm³/min'275° C.). Parent ions were detected on a full-scan model on Q1 quadrupole. Quantitative analysis was performed by selective reaction monitoring. The Q2 collision gas was argon at 1.5 mTorr, and daughter ions were detected on Q3. Selected parent/daughter ion pairs for NPD1 and free DHA were typically 359/205 m/z and 327/283 m/z, respectively. Calibration curves were obtained by running in parallel synthetic NPD1 and DHA (Cayman Chemical, Ann Arbor, Mich.).

NPD1 for Bioactivity Experiments. NPD1 was generated by biogenic synthesis using soybean lipoxygenase and DHA, purified by HPLC, and characterized by LC-MS/MS according to the above reported physical criteria.

RNA Isolation and Spectral Quality. Brain tissues were rapidly processed, and total RNA was extracted using Trizol reagent (Invitrogen, Carlsbad, Calif.). Total RNA quality and abundance were profiled using RNA Nano LabChips (Caliper Technologies, Mountainview, Calif.; Agilent Technologies, Palo Alto, Calif.).

Probe Synthesis and GeneChip Hybridization. Biotinylated antisense cRNAs were synthesized from cDNA using the Superscript Choice System (Invitrogen) and Enzo Biorray High Yield RNA Transcript Labeling kits according to the manufacturer's protocols (Affymetrix) and as described in Colangelo, V., Schurr, J., Ball, M. J., Pelaez, R. P., Bazan, N. G., Lukiw, W. J. (2002) J. Neurosci. Res., 70: 462-473; and Lukiw, W. J. (2004) Neurochem. Research, 29: 1287-1297. Probes were hybridized against HU133Av2 GeneChip DNA arrays that interrogate approximately 33,000 full-length mouse genes and expressed sequence tag (EST) clusters. Methodologies for first and second strand synthesis and conversion of double-stranded cDNA into biotinylated antisense cRNA, probe fragmentation, hybridization, washing and staining with streptavidin-R phycoerythrin (Molecular Probes, Eugene, Oreg.) and biotinylated goat anti-streptavidin antibody (Sigma Chemical; St. Louis, Mo.) have been previously described in Colangelo et al., 2002, and Lukiw, 2004.

GeneChip Data and Statistical Analysis. DNA arrays were scanned at 570 nm, and significant features were extracted using Data Mining Tool 3.0 (Affymetrix). Data analyses and graphic presentations were performed using Microarray Suite 5.0 (Affymetrix), GeneSpring 6.0 (Silicon Genetics, Redwood City, Calif.) and Adobe Photoshop 6.0 (Adobe Systems, San Jose, Calif.). Volcano plots were plotted representing total gene expression patterns as a function of fold-change as compared to expression patterns of age-matched controls as plotted against p, as calculated by an analysis of variance (ANOVA). (Data not shown) In this analysis, a stringent cut-off for genes was chosen that had a>2-fold change, either an increase or decrease over controls, and that achieved a significance level of p<0.05. Twelve HGU133 GeneChips (each containing 33,000 human gene targets) were used, three for each condition: (1) 3-week-old untreated control HN cells; (2) 3-week-old Aβ42 and DHA-treated HN cells; and (3) 3-week-old Aβ42 and NPD1-treated HN cells). The complete data for a select group of up- and down-regulated genes appear in Table 2. “Fold change” refers to comparison of the means of treated or control HN cells or AD and age-matched control brains. Statistical significance was analyzed using a two-way factorial analysis of variance (p, ANOVA; Statistical Analysis System; SAS Institute, Cary, N.C. ).

### EXAMPLE 8

### Effects of Cytokine Stressors on Human Neural Cells

Cytokine stressors were applied to developing human neural (HN) cells, a primary co-culture of neuronal and astroglial cells, over 8 weeks of development. HN cells were cultured for up to 8 weeks, and showed approximately equal populations of neurons and glia after 3 weeks of development. (Data not shown) The cells stained positive with the neuronal-specific markers NeuN and the glial-specific marker GFAP as described in Bazan, N. G., Lukiw, W. J. (2002) J. Biol. Chem., 277: 30359-30367. (Data not shown). HN cells under these conditions are a useful model for AD pro-inflammatory signaling.

To determine the effect of cytokine-mediated stress on aging HN cells, AP peptide release was assayed in the continuous presence in the culture medium of IL-1β, an inducer of reactive oxygen species (ROS) generation and oxidative stress as described in Lynch, A. M., Moore, M., Craig, S., Lonergan, P. E., Martin, D. S., Lynch, M. A. (2003) J. Biol. Chem., 278: 51075-51084; and Kaur, J., Dhaunsi, G. S., Turner, R. B. (2004) Med. Princ. Pract., 13: 26-29. Parallel experiments in aging HN cells were performed in the presence of control neuro-progenitor maintenance medium (NPMM) and the continuous presence of NPMM+DHA. Two neurotoxic forms of Aβ peptide, Aβ40, the predominant form of Aβ found in AD cerebrospinal fluid, and Aβ42, the peptide initially deposited within the neuritic plaque (which aggregates at a much lower concentration than Aβ40) were continuously monitored using Western immunoblot analysis of HN cell culture medium.

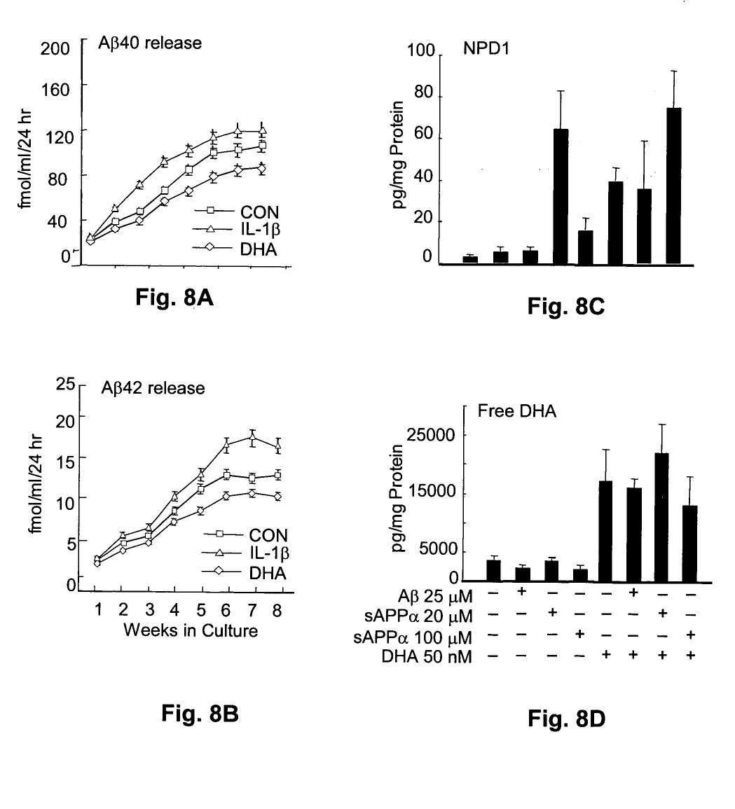

The secretion by HN cells of Aβ42 peptide is approximately one-tenth that of Aβ40 peptide in both control and treated cells as shown in FIGS. 8A and 8B. These results indicate that HN cells in culture secreted small, soluble Aβ peptides, whose concentrations increased as a function of aging, and that IL-1β enhanced Aβ peptide secretion while DHA suppressed this release. In contrast to cytokine-induced oxidative stress, DHA therefore elicited neuro-protective effects in cultured HN cells, in part, by decreasing the secretion of neurotoxic Aβ40 and Aβ42 peptides.

### EXAMPLE 9

### sAPPα Up-Regulates Neuroprotectin D1 Synthesis

HN cells were incubated as described above in the presence of sAPPα (sAPPA or sAPP; at 20 and 100 μM) and DHA (at 50 nM) to test the effects on concentrations of NPD1 and free DHA (FIGS. 8C and 8D). As described above, Aβ peptides are released into the cell culture medium from 1 to 8 weeks of culture as shown in FIGS. 8A and 8B. FIGS. 8C and 8D indicate that sAPPα at 100 μM induced NPD1 abundance to 80±7 pg mg⁻¹ protein, representing a mean 8-fold increase over untreated controls (N=6; p<0.05; FIG. 8C). This may be, in part, an effect of sAPPα on the NPD1 biosynthetic enzymes phospholipase A₂ (cPLA₂) and/or a 15-lipoxygenase-like enzyme. (See potential pathways in FIGS. 3 and 11D).

DHA/NPD1 may also have repressive effects on neurotoxic Aβ peptide production in cytokine- and oxidation-stressed brain cells. NPD1 induced neuroprotection via induction of the anti-apoptotic Bcl-2 family proteins Bcl-2 and Bcl-xL in oxidatively challenged retinal pigment epithelial cells as described below. Therefore, part of the neuroprotective effects of sAPPα, mediated through NPD1, may be through modulation of expression of Bcl-2 family members.

These data indicate that some of the neurotrophic effects of sAPPα are elicited, in part, by an up-regulation or increase in the biosynthesis of NPD1. They also indicate that neuroprotection could be enhanced by increasing the amount of NPD1 either by infusion of DHA or NPD1.

### EXAMPLE 10

### Neuroprotectin D1 Content is Reduced in CA1 Hippocampal Regions

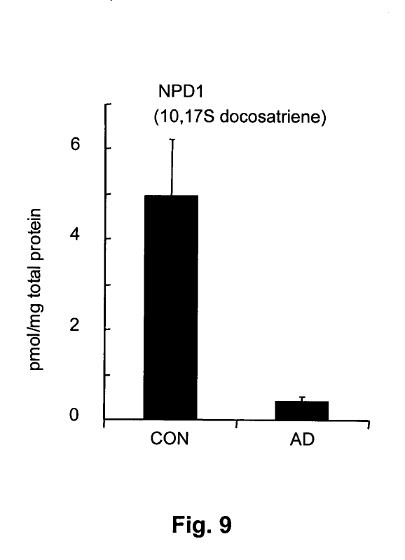

To establish the abundance of NPD1 in AD and age-matched control brains, tissues from the hippocampal CA1 region of 10 AD patients (5 female and 5 male) and 6 age-matched controls (3 female and 3 male) were used. NPD1 concentrations were determined using LC-MS/MS (sensitivity 0.05 pg mg⁻¹ total protein) as described above. As shown in FIG. 9, the reduction of NPD1 in AD versus control hippocampus reached a significance of p<0.02 (ANOVA).

In these experiments, free DHA pool size in controls measured on average 55.5±15.0 pg mg⁻¹ total protein. (Data not shown) In age-matched AD hippocampus, the DHA signal averaged less than half of that value, or 26.5±8.05 pg mg⁻¹ total protein. The high abundance of DHA, as an acyl side-chain of membrane phospholipids, suggests its importance in the brain as an essential component of normal brain function.

In controls, NPD1 averaged just 4.9±1.10 pg mg⁻¹ total protein. In age-matched AD, the NPD1 signal averaged less than one-tenth of that value or 0.42±0.20 pg mg⁻¹ total protein (FIG. 9B). The results indicate that, despite increased cPLA₂ activity in AD brain and perhaps as the result of excessive oxidative stress, both DHA and NPD1 are significantly reduced, and their neuroprotective effect during degenerative disease may have been effectively lost.

### EXAMPLE 11

### In AD Brain 15-LOX is Down-Regulated and Cytosolic PLA₂ is Up-Regulated

Although the precise enzymes involved in NPD1 synthesis have not been identified, mounting evidence suggests that a PLA₂ reaction followed by a 15-lipoxygenase-like enzyme are involved. (FIGS. 3 and 11D) Because both DHA and NPD1 were significantly down-regulated in AD brain when compared to healthy age-matched controls (FIG. 9; and Example 10), the levels of 15-LOX (GenBank M23892) and cPLA₂ (GenBank D38178; encoding an 82.5 kD calcium-dependent cytosolic PLA₂) were examined in 6 AD and 6 age-matched samples of hippocampal CA1 using DNA-array analysis employing HGU95Av2 GeneChips (13,000 gene targets; Affymetrix, Santa Clara, Calif.). The status of 15-LOX and cytosolic PLA₂ in AD hippocampal CA1 is shown in Table 1. In Table 1, (CON=control; AD=Alzheimer) the numbers 1-6 indicate an individual case number; “plaque-tangle count” refers to average lesion density mm⁻², and ranges and means±one standard deviation for all data are shown. When compared to age-matched controls, 15-LOX is reduced in AD about 2.1-fold (p<0.05), and cPLA₂ abundance is increased in AD 4.5-fold (p<0.02).

TABLE 1
Changes in 15-LOX and cPLA₂ Gene Expression in Control and AD Brain
Hippocampus

| | | | | | | | |
|---|---|---|---|---|---|---|---|
| | | | | Plaque-tangle | | 15-LOX | cPLA₂ |
| Case | Age | Sex | PMI (hr) | count | RNA | (M23892) | (D38178) |

CONTROLS

| | | | | | | | |
|---|---|---|---|---|---|---|---|
| CON1 | 70 | M | 1.3 | 0/5 | 2.0 | +1634 | +856 |
| CON2 | 69 | M | 3.0 | 0/2 | 2.1 | +1094 | +2111 |
| CON3 | 68 | F | 2.0 | 1/2 | 1.9 | +102 | +1220 |
| CON4 | 71 | F | 1.5 | 0/4 | 2.1 | +1235 | +1810 |
| CON5 | 66 | F | 2.4 | 0/5 | 2.0 | +1150 | +1743 |
| CON6 | 70 | M | 2.5 | 1/2 | 1.9 | +950 | +1338 |
| Range | 66-71 | 3F/3M | 1.3-3.0 | — | — | +102 to 1634 | +856 to +2111 |
| mean ± SD | 69.0 ± 1.8 | | 2.1 ± 0.6 | — | — | +1027 ± 508 | +1513 ± 458 |

ALZHEIMER'S PATIENTS

| | | | | | | | |
|---|---|---|---|---|---|---|---|
| AD1 | 68 | F | 1.5 | 8/15 | 2.0 | −1131 | +8324 |
| AD2 | 72 | M | 2.3 | 6/13 | 2.0 | −404 | +1870 |
| AD3 | 70 | F | 1.3 | 7/12 | 2.0 | −1944 | +9568 |
| AD4 | 67 | M | 2.1 | 6/14 | 2.1 | −211 | +8566 |
| AD5 | 69 | F | 1.6 | 8/10 | 2.0 | −704 | +4358 |
| AD6 | 76 | M | 3.0 | severe | 1.9 | −1442 | +9020 |
| range | 67-76 | 3F/3M | 1.3-3.0 | — | — | −1944 to −211 | +1870 to +9568 |
| mean ± SD | 70.3 ± 3.3 | | 2.0 ± 0.6 | — | — | −973 ± 557 | +6951 ± 2712 |

As shown in Table 1, there is a down-regulation in the abundance of 15-LOX and an up-regulation in the abundance of cPLA₂ in AD brain. These enzymes may be responsible for the biosynthesis of DHA and NPD1, as explained above. The decreased abundance of neurotrophic DHA/NPD1 in AD brain may therefore be explained, in part, by a disruption in the activity of the enzymes 15-LOX and cPLA₂ essential for their biosynthesis. DHA/NPD1 generation may thus be impaired during the normal aging of brain cells in culture and in the normal aging of the brain as observed in AD.

### EXAMPLE 12

### NPD1 Up-Regulates the Expression of Anti-Apoptotic Bcl-2 Proteins

A DNA array-based survey was performed using human genome HGU133 GeneChips (Affymetrix) on the effects of NPD1 or DHA in HN cells in primary culture. The analysis focused on the levels of gene expression for the pro-inflammatory cytokines IL-1β and CEX-1, COX-2, B94, and TNFα, whose levels are known to be significantly up-regulated in AD tissues; and five members of the Bcl-2 gene family, three of which are anti-apoptotic (Bcl-2, Bfl-1 [A1], and Bcl-xL) and two of which are pro-apoptotic (Bax and Bik). See, Colangelo et al., 2002; Lukiw, 2004; and Akhtar, R. S., Ness, J. M., Roth, K. A. (2004) Biochim. Biophys. Acta., 1644: 189-203.

HN cells were treated with either Aβ42 (25 μM), DHA (50 nM), or NPD1 (50 nM) for 18 h. In the DNA analysis, two stringent criteria for cut-off were used: (1) changes in gene expression achieving an experimental significance of p<0.05 (ANOVA); and (2) changes of 2-fold or greater over controls, either up- or down-regulated. A “volcano plot’ representation of these data for NPD1-treated HN cells was generated using GeneSpring v6.1 algorithms (Silicon Genetics). (Data not shown).

Ten of the most significant gene-expression changes are outlined in Table 2. This DNA-array analysis indicated up-regulation of Bcl-2 and Bfl-1(A1) and relative down-regulation of Bax and Bik, neuroprotective and pro-apoptotic members of the Bcl-2 gene family, respectively (Table 2). In this analysis Bax and Bik attained levels of up-regulation of +3.2 and +2.8 (p<0.05) in Aβ42-treated cells over age-matched control cells. These enhanced levels were driven to the status of no significant change (NSC) after treatment with DHA or NPD1. Both anti-apoptotic Bcl-2 and Bfl-1(A1) gene expressions were significantly increased after DHA and NPD1 treatment (Table 2). The anti-apoptotic Bcl-2 family member Bfl-1(A1) was up-regulated by both DHA and NPD1 to 3.9- and 6.7-fold over controls, respectively; representing one of the most significantly up-regulated genes in NPD1-treated HN cells.

In Table 2, fold change increases for Aβ42, DHA, and NPD1 are expressed as fold changes over untreated controls. All positive (+) values in this analysis achieved fold change status of 2-fold or greater over untreated controls and were highly significant (p<0.05). CEX-1 (GenBank U64197), “Chemokine exodus protein 1,” is a marker for the presence of inflammatory and oxidative stress response. See, Colangelo et al., 2002 and Lukiw, 2004. Bik (GenBank BC001599) is a pro-apoptotic protein. See, Metcalfe, A. D., Hunter, H. R., Bloor, D. J., Lieberman, B. A., Picton, H. M., Leese, H. J., Kimber, S. J., Brison, D. R. (2004) Mol. Reprod Dev., 68: 35-50. B94 (GenBank M92357) is a TNFα-inducible pro-inflammatory element. See, Colangelo et al., 2002. The gene transcripts have been classified according to main function(s); however, they have additional roles.

TABLE 2
Changes in Gene-Expression Patterns in Aβ42-, DHA-,
or NPD1-Treated HN Cells.
Analytical criteria were >2.0 fold over control;
and p < 0.05, ANOVA; nsc = no significant change.

| | Function | | | | | |
|---|---|---|---|---|---|---|
| | Apoptotic | | | | | |
| Gene | Pro | Anti | Inflammatory | Aβ42 | DHA | NPD1 |
| IL-1β | + | + | + | +4.1 | nsc | nsc |
| CEX-1 | + | + | + | +5.7 | nsc | nsc |
| Bfl-1 (A1) | − | + | − | nsc | +3.9 | +6.7 |
| Bcl-2 | − | + | − | nsc | +2.5 | +3.3 |
| Bcl-xL | − | + | − | nsc | +1.5 | +2.4 |
| Bax | + | − | − | +3.2 | nsc | nsc |
| Bik | + | − | − | +2.8 | nsc | nsc |
| COX-2 | + | − | + | +5.4 | nsc | nsc |
| B94 | + | − | + | +4.2 | nsc | nsc |
| TNFα | + | − | + | 4.8 | Nsc | nsc |

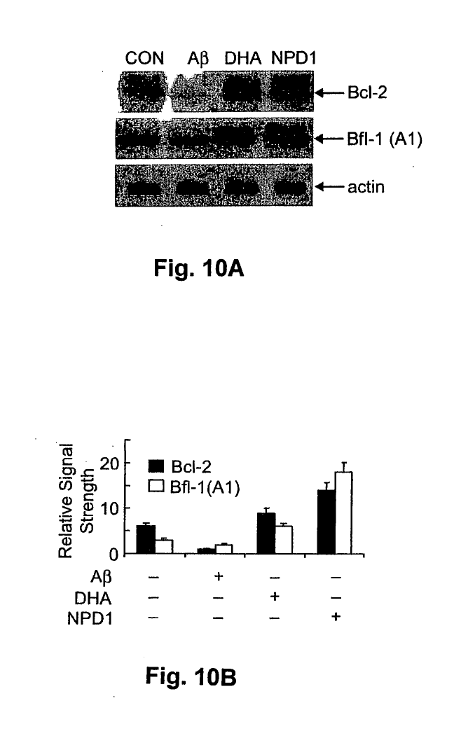

To confirm the above changes in Bcl-2 and Bfl-1(A1), Western analysis was performed using anti-Bcl-2, anti-Bfl-1(A1), and anti-actin primary antibodies (B3170 Sigma Life Science, sc-8351 Santa Cruz, and A2103 Sigma, respectively). The results of the Western analysis confirm up-regulation of Bcl-2 and Bfl-1(A1) anti-apoptotic proteins over actin controls in DHA- and NPD1-treated cells. FIG. 10B shows quantification of the intensity of the Bcl-2 and Bfl-1(A1) bands (from 30 μg HN protein extract) as shown in FIG. 10A, normalized with constitutively expressed actin (from 10 μg HN protein using the same extract and membrane). Bcl-2 and Bfl-1(A1) after NPD1 treatment showed significant up-regulation, averaging 2.3-fold and 5.4-fold increases, respectively, over controls (each p<0.05).

Neurotrophism and neuronal survival are probably not controlled by single genes, but by multiple genes or gene families. The data in Table 2 for the Bcl-2 gene family show that DHA/NPD1 induces a gene-expression program that is largely neuroprotective, by down-regulation of pro-apoptotic and pro-inflammatory factors and up-regulation of Bcl-2 anti-apoptotic proteins that are critical regulators of neuronal cell survival. These results again show that NPD1 was much more effective that DHA in upregulating these neuroprotective anti-apoptotic genes.

Protection for Retinal Pigment Epithelial Cells

We have discovered that retinal pigment epithelial cells synthesize 10,17S-docosatriene (NPD1) from DHA. Human retinal pigment epithelial cells were used to demonstrate the synthesis of 10,17S-docosatriene (neuroprotectin D1, NPD1), a stereospecific mediator derived from endogenous DHA. This synthesis was enhanced by IL-1β, as well as by supplying DHA to the culture medium. NPD1 and DHA potently counteracted H₂O₂/TNFα oxidative stress-mediated apoptotic RPE damage. Under the same oxidative-stress conditions, NPD1 up-regulated the anti-apoptotic Bcl-2 proteins, Bcl-2 and Bcl-xL, and decreased expression of the pro-apoptotic proteins, Bad and Bax. Moreover, NPD1 inhibited oxidative stress-induced caspase-3 activation and inhibited IL-1β-stimulated human COX-2 promoter. NPD1 also inhibited apoptosis in RPE cells due to A2E. Overall, NPD1 protected retinal pigment epithelial cells from oxidative stress, and contributed to photoreceptor cell survival.

### EXAMPLE 13

### Materials and Methods for Retinal Pigment Epithelial Cells

ARPE-19 cell culture and chemicals: ARPE-19 cells (L. M. Hjelmeland, ATCC # CRL-2302) were grown and maintained in DMEM-F12 medium supplemented with 10% FBS and incubated at 37° C. with a constant supply of 5% CO₂. ARPE-19 cells are spontaneously transformed human retinal pigment epithelial cells that conserve cellular biological and functional properties. All chemicals were purchased from Sigma Chemical Co. (St. Louis, Mo.) unless otherwise indicated.

LC-MS-MS analysis, synthesis, and characterization of 10,17S-docosatriene (NPD1). ARPE-19 lipid extracts were spiked with deuterated internal standards, then purified by solid-phase extraction. Samples were analyzed as described above. Samples were pre-equilibrated at pH 3.0, loaded to C18 columns (Varian), and eluted with 10 ml 1% methanol in ethyl acetate (EM Science). See, Marcheselli, V. L. Hong, S., Lukiw, W. J., Tian, X. H., Gronert, K., Musto, A., Hardy, M., Gimenez, J. M., Chiang, N., Serhan, C. N. & Bazan, N. G. (2003) J. Biol. Chem. 278:43807-43817; Erratum in: J. Biol. Chem. (2003) 278:51974. Samples were concentrated on N₂ stream evaporator before LC-MS analysis. Samples were then loaded on a Biobasic-AX column (Thermo-Hipersyl-Keystone) (100 mm×2.1 mm, 5-μm particle sizes). The column was run with a 45-min gradient protocol, starting with solvent solution A (40:60:0.01 methanol/water/acetic acid, pH 4.5), at a flow rate of 300 μl/min; the gradient reached 100% solvent B (99.99:0.01 methanol/acetic acid) in 30 min, and then was run isocratically for 5 min. The system returned to 100% solvent solution A in 10 min. After running through a PDA detector (Surveyor, ThermoFinnigan, San Jose, Calif.) set on scan mode at wavelength range of 200-600 nm, LC effluents were diverted to an electro-spray-ionization probe (ESI) on a TSQ Quantum (ThermoFinnigan) triple quadrupole mass spectrometer. To generate molecular ionization, the ESI probe was set at a spray voltage of 3 kv, N₂ as a sheath gas at a flow rate of 35 cm³/min, and capillary temperature of 275° C. Parent ions were detected on full-scan mode on Q1 quadrupole. For quantitative analysis, selected ion monitoring (SIM) was performed, filling Q2 collision chamber with argon gas at 1.5 mTorr, and daughter ions detected on Q3. The selected parent/daughter ion pairs for NPD1 and free DHA were 359/205 m/z and 327/283 m/z, respectively. Calibration curves were obtained running in parallel with synthetic NPD1 (prepared as described below) and DHA (Cayman Chemicals, Ann Arbor, Mich.). NPD1 for bioactivity studies was generated by biogenic synthesis using soybean lipoxygenase and DHA and characterized by LC-PDA-MS-MS according to the reported physical criteria.

Transfection of ARPE-19 cells with human COX-2 (830 bp): ARPE-19 cells growing in 12-well plates were transfected with 5 μg human COX-2-luciferase construct (830 bp) by FuGENE-6 (obtained from Drs. S. Prescott and D. Dixon, Vanderbilt University, Nashville, Tenn.). Four hours later, the medium containing FuGENE-6 was removed, fresh DMEM-F12 (10% FBS) was added, and the cells further incubated 8 h at 37° C. Transfected cells were serum-starved for 4 h and challenged with IL-1β (10 ng/ml) for 8 h with or without NPD1. NPD1 was added at concentrations from 0.05 to 100 nm. Eight hours later, cell homogenates were made, protein concentrations were adjusted, and luciferase assays were performed using 20 μg equivalent protein in Monolight 2010 for 20 seconds with luciferin as substrate, as described below.

Luciferase assay: The medium containing IL-1β and NPD1 was removed, and the cells were washed with 1 ml cold PBS. The cells were scraped and centrifuged 2500×g for 20 minutes at 4° C. The cell pellet was re-suspended in 500 μl of 2×luciferase assay buffer (ALL, 0.2 mM tricine, 2 mM DTT, 30 mM MgSO₄ and 10 mM ATP). Cells were lysed and cellular debris was pelleted by centrifugation at 1200×g. A 20-μg protein sample was used in each assay and mixed with 70-80 μl 2×ALL buffer. The reaction was initiated by the injection of 100 μl of 1 mM luciferin. The relative light units were determined by using an ALL luminometer recording over a 20-second interval, as described in Bazan, N. G., Fletcher, B. S., Herschman, H. R. & Mukherjee, P. K. (1994) Proc. Natl. Acad. Sci. U.S.A. 91:5252-5256. The luciferase assays were performed on triplicate plates and normalized for protein content with the Bio-Rad protein assay kit.

DHA supplementation to ARPE-19 cells for intracellular accumulation of NPD1: Two approaches were adopted. In the first approach, 80% confluent ARPE-19 cells were serum-starved for 1 h then treated with TNFα (10 ng/ml) plus H₂O₂ (0.3 μM) for 4 h. Cells were harvested in 0.5 ml methanol, and lipids were purified and extracted by solid-phase extraction for mass spectrometry. In the second approach, DHA (6.7 μM) in BSA (3.35 μM) was added during the plating of the ARPE-19 cells. The cells were allowed to grow in the presence of DHA for 72 h, after which cells were serum-starved for 1 h and induced with either IL-1β or TNFα plus H₂O₂. Four hours later, cells were harvested, extracted in 0.5 ml methanol, and treated as above.

DNA fragmentation assay: Cells (80% confluent) growing in DMEM (10% FBS) in 6-well plates were labeled with 1 μCi ³H-thymidine per well for 24 h at 37° C. The day-old medium was replaced to remove the unincorporated ³H-thymidine. Cells were then serum-starved for 1 h and treated with TNFα plus H₂O₂ for 6-8 h. Cells were harvested, centrifuged at 200×g for 10 min at 4° C. An aliquot of the supernatant was precipitated with 25% TCA, and radioactivity was measured. This represents the amount of ³H-thymidine released during the oxidative stress induced by TNFα plus H₂O₂ The cell pellet was solubilized in lysis buffer (0.2% Triton X-100 in 10 mM Tris/EDTA). The intact DNA and fragmented DNA were separated by centrifugation at 13,000×g for 10 min at 4° C. Fragmented DNA was precipitated from the supernatant with 25% TCA. The pellets were resuspended in 1% SDS and radioactivity was measured. This represents the intact DNA in the cells. The amount of DNA fragmentation is expressed as percentage of fragmented DNA over the control, as described in Kim, H. Y., Akbar, M., Lau, A. & Edsall, L. (2000) J. Biol. Chem. 275:35215-35223.

Assessment of apoptosis by mono- and oligonucleosome analysis: Cells were homogenized in a lysis buffer and 20-μl aliquots were applied on streptavidin-coated 96-well plates. Cells were incubated for 2 h with 80 μl incubation buffer containing the monoclonal antibodies directed against DNA and histones for detection of mono- and oligonucleosomes (Roche Diagnostics Corporation Cat. # 1774425). After the unreacted antibodies were washed away, the immune complexes of DNA-histone-antibodies remain bound to the streptavidin-coated plates, where detection was attained by horseradish-peroxidase reaction.

Analysis of pro- and anti-apoptotic proteins in ARPE-19 cells: Bcl-2 family proteins were analyzed by Western-blot analysis. In brief, 20-μg equivalents of each cell extract were subjected to electrophoresis on a 8-16% gel (Promega, Madison, Wis.) at 125 volts for 2 h. The proteins were transferred to nitrocellulose membrane at 30 volts for 1 h at 4° C. The membranes were subjected to treatment with primary antibodies of Bcl-2, Bcl-xL, Bax, and Bad for 1 h at room temperature, and probed 30 min with secondary antibody, goat anti-rabbit Ig:HRPO and HRP-conjugated anti-biotin antibody. (BD Sciences—Pharminogen, San Diego, Calif.) Then proteins were evaluated using an ECL kit. (Amersham Biosciences Corp., Piscataway, N.J.)

Caspase-3 cleavage analysis: Caspase-3 cleavage was evaluated by Western-blot analysis after using PARP as substrate. Briefly, 20 μg equivalent of each cell extract was electrophoresed on an 8-16% gel (Promega, Madison, Wis.) for 2 h at 125 volts. The proteins were transferred onto nitrocellulose membranes as before, and probed with PARP antibody (Santa Cruz Biotechnology, Santa Cruz, Calif.). The degradation of PARP was evaluated using ECL kit.

Hoechst staining: ARPE-19 cells were loaded with 2 μM Hoechst dissolved in a Locke's solution (Promega, Madison, Wis.) and incubated one h at 37° C. before imaging. Cells were then washed once with PBS and viewed using a Nikon DIAPHOT 200 microscope under UV fluorescence. Images were recorded by a Hamamatsu Color Chilled 3CCD camera and Photoshop 5.0 software, as described in Mukherjee, P. K., DeCoster, M. A., Campbell, F. Z., Davis, R. J. & Bazan, N. G. (1999) J. Biol. Chem. 274:6493-6498.

### EXAMPLE 14

### 10,17S-docosatriene (NPD1) Generation by Human RPE Cells.

Human RPE cells in culture contain detectable levels of 10,17S-docosatriene (NPD1), as shown in FIG. 11A. FIG. 11A represents a MS-MS spectrum for NPD1: full scan of negative ion products for selected parent ion (m/z 359). The insert depicts a UV spectrum for NPD1. The MS-MS pattern of this lipid indicated that it represented a dihydroxy-docosahexaenoic acid with a base peak[M-H] ion m/z 359, and with fragment ions at m/z 341, 297, 289, 261, 243, 205, 181, and 153. Diagnostic ions were essentially identical to those recently described in other tissues.

IL-1β stimulated the release of DHA and the synthesis of NPD1 (FIG. 11B) in a time dependent fashion. FIG. 11B indicates the increased production of 10,17S-docosatriene (NPD1) by IL-1β (10 ng/ml) after 6 h incubation. As described in Example 13, cells at confluence were serum-starved then treated for 6 h with IL-1β before lipid extraction and LC-MS-MS analysis.

Furthermore, ARPE-19 cells stimulated with the calcium ionophore A-23187 (10 μM) displayed a time-dependent increase of free DHA and NPD1 (FIG. 11C). DHA and NPD1 were measured as above by LC-MS-MS analysis. This experiment was performed without adding exogenous DHA, and demonstrated a 3- to 4-fold higher free DHA pool size available as compared to the amount of NPD1 formed. Thus activation of a phospholipase A₂ that releases free DHA is an event that precedes NPD1 synthesis.

FIG. 11D depicts a possible NPD1 biosynthetic pathway. After phospholipase A₂ releases DHA, a lipoxygenase-like enzyme catalyzes the synthesis of 17S-H(p)DHA, which in turn is converted to a 16(17)-epoxide that is enzymatically converted to NPD1. Early evidence of both the enzymatic synthesis of docosanoids in the retina as well as the formation of a 17S-H(p)DHA preceding the synthesis of 10,17S-docosatriene (NPD1) in brain ischemia-reperfusion has been reported. See, Marcheselli, V. L. Hong, S., Lukiw, W. J., Tian, X. H., Gronert, K., Musto, A., Hardy, M., Gimenez, J. M., Chiang, N., Serhan, C. N. & Bazan, N. G. (2003) J. Biol. Chem. 278:43807-43817. Erratum in: J. Biol. Chem. (2003) 278:51974.

### EXAMPLE 15

### Oxidative Stress-Triggered Apoptosis in Human RPE Cells is Inhibited by NPD1.

Several experimental conditions that result in reproducible oxidative-stress-induced apoptosis in ARPE-19 cells were tested. One condition chosen was the combination of TNFα with H₂O₂, both in relatively low concentrations. Typically 10 ng/ml of TNFα and 400-800 μM H₂O₂ led to marked increase in Hoechst-positive cells (Data not shown). Neither TNFα nor H₂O₂ alone triggered a significant effect under these conditions. (Data not shown).

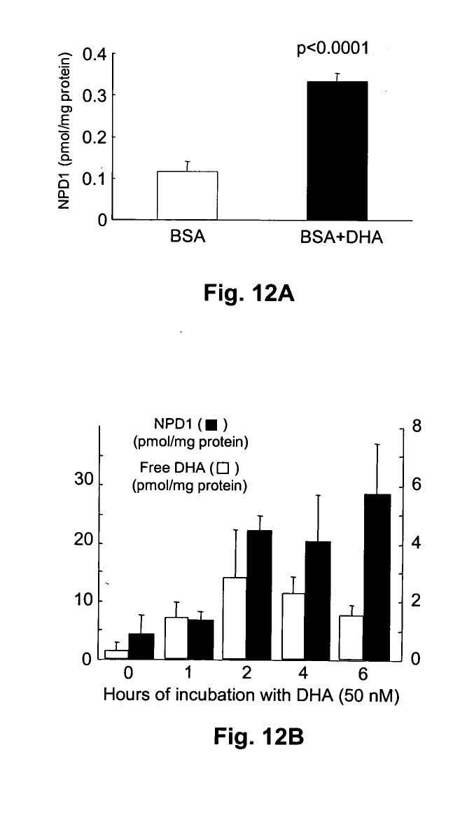

Serum starvation made ARPE-19 cells more sensitive to oxidative stress-triggered apoptosis induced by TNFα/H₂O₂. Cells were grown in culture for 72 h in the presence of BSA (3.35 μM) with or without DHA (6.7 μM). Endogenous NPD1 formation was enhanced by BSA-DHA (FIG. 12A). When these cells were serum-starved for 1 h and then exposed for 14 h to TNFα/H₂O₂ during serum starvation, a greatly reduced number of Hoechst-positive cells were observed as a result of the pre-incubation with BSA-DHA (Data not shown). This experiment indicates that NPD1 synthesized in the ARPE-19 cells exerted its protective action at an early event of the apoptotic cascade.

In the next experiment, confluent cells were starved for 8 h, then TNFα/H₂O₂ was added and incubated for 14 h before staining with Hoechst reagent. NPD1 (50 nM) was added at the time of adding TNFα/H₂O₂. This NPD1 addition resulted in an inhibition of oxidative stress induction of Hoechst-positive cells (Data not shown). When the TNFα concentration (10 ng/ml) was maintained and H₂O₂ concentration was decreased from 800 to 400 μM, fewer Hoechst-positive cells were seen. NPD1 (50 nM) almost completely inhibited the oxidative stress caused by the lower H₂O₂ concentration. Moreover, this effect seems to be rather specific, since neither PGE₂, LTB₄, nor 20-OH-LTB₄ (each at 50 nM) was able to elicit the degree of inhibition due to NPD1, although LTB₄ was partially inhibitory. (Data not shown).

Additional experiments were conducted that compared the effect of arachidonic acid (50 nM) or DHA (50 nM) added to the culture medium on oxidative stress-induced apoptosis. Arachidonic acid was partially effective, but DHA proved to be a very potent inhibitor. (Data not shown) Therefore, an experiment was designed to test whether the inhibitory activity of added DHA might be correlated with NPD1 formation. FIG. 12B shows the time course of formation of NPD1 in RPE cultures treated with DHA (50 nM). Synthesis of NPD1 occurred as a function of incubation time. The free DHA content rose as a function of incubation time up to 2 h, then decreased. These changes in DHA pool size during serum starvation (FIG. 12B) contrasted with those seen in FIGS. 11B and 11C, without serum starvation. In FIGS. 11B and 11C, stimulation by IL-1β or calcium ionophore promoted a sustained release of endogenous DHA from membrane phospholipids.

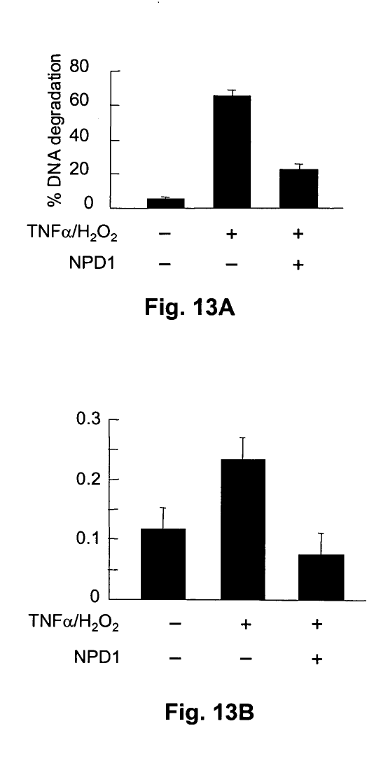

These observations indicate that oxidative stress triggered DNA fragmentation and RPE apoptosis, and that the increase in NPD1 inhibited RPE cell death. Two additional approaches to assess apoptosis were used under these same conditions: (1) DNA fragmentation by differential sedimentation after ³H-thymidine labeling (FIG. 13A), and (2) ELISA detection of mono- and oligonucleosomes (FIG. 13B). To detect DNA fragmentation by the first method, ARPE-19 cells were prelabeled with ³H-thymidine for 24 h. Medium was removed and cells were washed with serum-free medium, serum-starved for 1 h, then treated with TNF-α (10 ng) and H₂O₂ (800 μM) for 6-8 h in the presence or absence of NPD1 (50 nM) to induce oxidative stress. DNA fragmentation was evaluated after TCA precipitation and expressed as percentage over intact DNA. The data presented in FIG. 13A are representative of two independent experiments, each of which was performed using quadruplicate cultures.

DNA fragmentation was also detected by ELISA detection of mono- and oligonucleosomes in ARPE cells (Roche Diagnostics Corporation Cat. # 1774425). Cells were homogenized in a lysis buffer, and 20-μl aliquots were applied on streptavidin-coated 96-well plates. Cells were incubated for 2 h with 80 μl incubation buffer containing the monoclonal antibodies directed against DNA and histones for detection of mono- and oligonucleosomes. After the unreacted antibodies were washed away, the immune complexes of DNA-histone-antibodies remained bound to the streptavidin-coated plates, where detection is obtained through horseradish-peroxidase reaction. Quantitative analysis was performed in a Spectramax-250 (Molecular Devices) spectrophotometer.

TNFα/H₂O₂ produced a marked DNA degradation as assessed by both of these methods (FIGS. 13A,B). Moreover NPD1 inhibited DNA fragmentation, as indicated by inhibition of DNA degradation by direct assessment using ³H-thymidine (FIG. 13A). In addition, the TNFα/H₂O₂-induced accumulation of mono- and oligonucleosomes was inhibited by NPD1, further indicating that oxidative stress-triggered DNA breakdown is attenuated by the DHA-derived mediator (FIG. 13B).

### EXAMPLE 16

### NPD1 Stimulated Anti-Apoptotic Bcl-2 Protein Expression and Decreased Pro-Apoptotic Protein Expression During Oxidative Stress

Since Bcl-2 family proteins participate in the initiation and amplification of premitochondrial events in the apoptosis cascade, their participation was investigated in TNFα/H₂O₂-induced ARPE-19 cell death as well as the possibility that they are a target for NPD1 action. Two different concentrations of H₂O₂ (400 or 800 μM) plus TNFα (10 nM) were studied, which resulted in proportionally different numbers of Hoechst-positive cells. In both instances added NPD1 inhibited apoptosis (Data not shown).

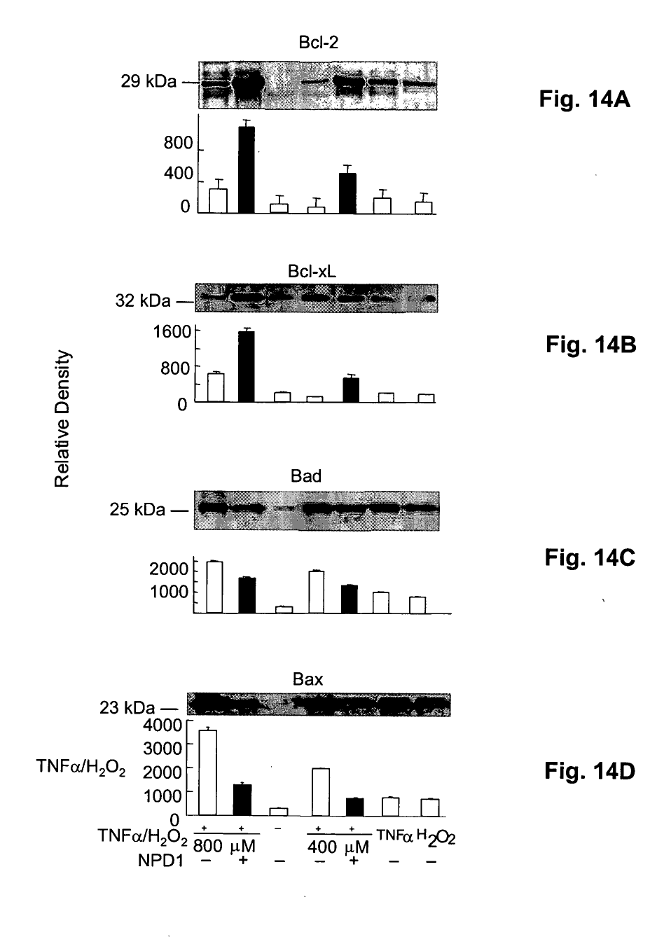

Using the same TNFα/H₂O₂ concentrations, Bcl-2 proteins were assayed. Cells were grown for 72 h after plating, placed in serum-free medium for 1 h, then incubated with TNFα/H₂O₂ for 6 h. Protein expression and Western-blot analysis were performed as described in Example 13. Data in FIGS. 14A, 14B, 14C and 14D represent four independent experiments with triplicate samples in each case. Anti-apoptotic protein Bcl-xL was enhanced in ARPE-19 cells by TNFα/H₂O₂ (800 μM) (FIG. 14B, first bar), whereas Bcl-2 was not changed (FIG. 14A, first bar). Addition of NPD1 increased expression of both Bcl-xL and Bcl-2 (FIGS. 14A and 14B; second and fifth bars).

The pro-apoptotic proteins, Bax and Bad, were up-regulated by TNFα/H₂O₂ (800 μM) by 7- and 5.3-fold, respectively. (FIGS. 14C and 14D, first bar) A much lower Bax up-regulation was observed with 400 μM H₂O₂ plus TNFα (FIG. 14D), than with 800 μM H₂O₂. However, Bad showed similar responses to both concentrations of H₂O₂ in combination with TNFα (FIG. 14C). In addition, H₂O₂ or TNFα alone somewhat increased Bax and Bad basal levels. When added in the presence of TNFα/H₂O₂, NPD1 decreased the up regulation of Bax by 65% using 800 μM H₂O₂ along with TNFα (FIG. 14D, second and fifth bars). However, the effect of the NPD1 on oxidative stress-increased Bad was smaller, only a 52% decrease under similar conditions. (FIG. 14C, second and fifth bars).

### EXAMPLE 17

### Oxidative Stress-Mediated Caspase-3 Cleavage is Attenuated by NPD1.

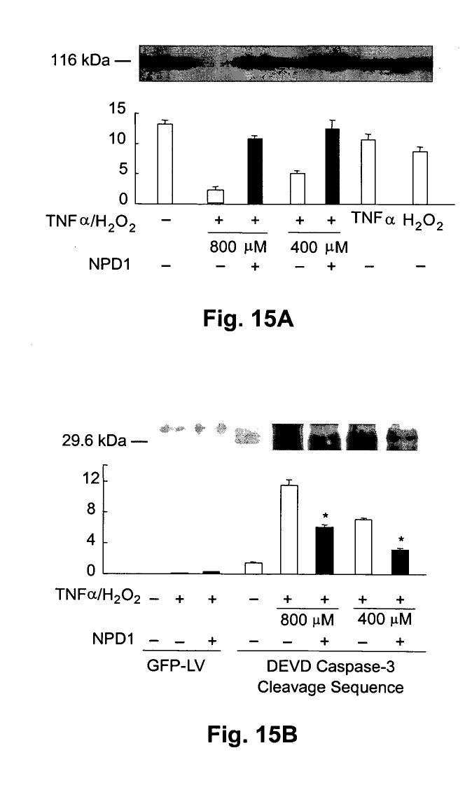

Effector caspase-3, downstream of pro- and anti-apoptotic proteins, is activated as a consequence of mitochondrial cytochrome c release into the cytoplasm and activation of the apoptosome. ARPE-19 cells were serum-starved for 1 h prior to a 6- to 8-h treatment with TNF-α (10 ng)+H₂O₂ (either 400 or 800 μM as indicated) in the presence or absence of NPD1 (50 nM). In FIG. 15A, caspase-3 cleavage was detected by Western-blot analysis using PARP (Santa Cruz Biotech.) as antibody. The values are the average of triplicate samples in three independent experiments. In ARPE-19 cells, caspase-3-induced cleavage using endogenous substrate occurred when TNFα/H₂O₂-triggered oxidative stress was present, as indicated by increased PARP accumulation (an 80.8% increase compared to the control; FIG. 15A). In addition, NPD1 was able to protect the caspase-3 activation when added with TNFα/H₂O₂ (18.3% increase compared to the control); TNFα or H₂O₂ alone elicits little caspase-3 activation (15.9% or 29.4% increase compared to the control).

To further ascertain the involvement of caspase-3, ARPE-19 cells were transduced with a construct encoding a peptide containing the caspase-3 cleavage sequence DEVD using a lentivirus (obtained from Drs. P. Nicotera and D. Bano, University of Leicester, Leicester, United Kingdom). This approach resulted in stably transfected cells. FIG. 15B shows the cleavage of caspase-3 during oxidative stress-induced apoptotic conditions using both 800 and 400 μM H₂O₂ in combination with TNFα. The values shown are the average of triplicate samples in three independent experiments. NPD1 inhibited caspase-3 activation by 50 and 60%, respectively.

### EXAMPLE 18

### IL-1β-Induced COX-2 Expression is Inhibited by NPD1

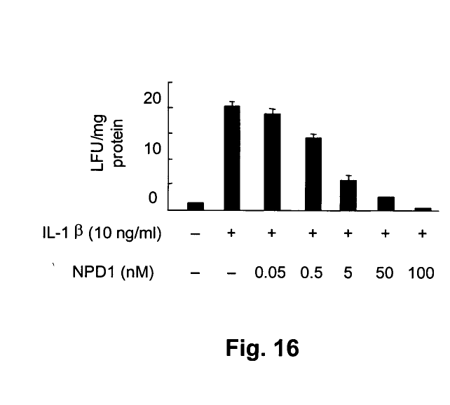

Pro-inflammatory gene expression is enhanced during oxidative stress and represents an important route of cell injury under these conditions. Cyclooxygenase (COX)-2 is an inducible enzyme that catalyzes the synthesis of prostaglandins and that is involved in oxidative stress as well as in cell function. COX-2 is actively regulated in the RPE. See, Ershov, A. V. & Bazan, N. G. (1999) J. Neurosci. Res. 58:254-261. Therefore, ARPE-19 cells were transfected with a 5′ deletion construct of the human COX-2 promoter containing 830 bp fused to a luciferase reporter gene. IL-1β induces a prominent increase in COX-2 promoter expression, as shown in FIG. 16. In a dose-dependent fashion, NPD1 potently counteracted the cytokine induction of the COX-2 promoter. The results presented in FIG. 16 indicate that under these conditions, NPD1 displays an IC₅₀ below 5 nM.

### EXAMPLE 19

### NPD1 Counteracts A2E Induced Oxidative Stress in Human Retinal Pigment Epithelial Cells.

N-retinylidene-N-retinylethanolamine (A2E), a hydrophobic fluorophore from lipofuscin, accumulates in relatively high concentrations in retinal pigment epithelial cells in Stargardt's disease and other retinal degenerations. In fact, A2E triggers damage and cell death in the RPE.

To test the ability of NPD1 to inhibit A2E oxidative stress, ARPE-19 cells grown in 6-well plates were transfected by Fugene 6 with NF-κB promoter sequence linked to a luciferase reporter gene and with IκB-EGFP. Promoterless β-galactosidase was co-transfected in ARPE-19 cells to determine the transfection efficiency. Four hours after transfection, cells were washed and further incubated 8 to 10 h before adding inducers. Two different set of cells were used. One set of cells was serum-starved 5 h before the addition of A2E and TNF-α/H₂O₂, and further incubated for 20-24 h before harvesting. The other set of cells was not serum-starved or further incubated. Luciferase activity was measured. The IκB-EGFP probe was measured by fluorescence confocal microscopy, and Hoechst-positive cells were identified.

A2E enhanced oxidative stress in ARPE cells. (Data not shown) A2E at concentrations up to 200 μM induced NF-κB and apoptosis as shown by Hoechst staining. Transfection with IκB-EGFP resulted in up-regulation of NF-κB after 20 h. Hoechst staining of A2E-induced ARPE-19 cells indicated apoptotic cell death by the same time. However, NPD1 inhibited A2E-induced apoptosis.

NPD1 inhibited A2E-mediated oxidative stress in ARPE-19 cells. Therefore, NPD1 may be useful as a cytoprotective approach in age-related macular degeneration, Stargardt's disease, and related conditions.

The above results demonstrated that ARPE-19 cells synthesize 10,17-S-docosatriene (neuroprotectin D1, NPD1), a stereospecific mediator from docosahexaenoic acid (DHA) oxygenation. In addition, NPD1 was shown to be a potent inhibitor of oxidative stress-induced apoptosis (and of cytokine-triggered pro-inflammatory COX-2 gene promoter induction).

Bcl-2 family proteins regulate apoptotic signaling at the level of the endoplasmic reticulum and mitochondria. As a consequence, cytochrome c is released from mitochondria and effector caspase-3 is activated. In agreement with this sequence, the above data confirm that oxidative stress activated caspase-3 in ARPE-19 cells. NPD1 (50 nM) decreased the activation of caspase-3 caused by oxidative stress. Moreover, apoptosis was an outcome of TNFα/H₂O₂-induced oxidative stress in ARPE-19 cells. Interestingly, NPD1 was effective in counteracting ARPE-19 cell death.

Collectively these findings demonstrated that neuroprotectin D1 (NPD1; 10,17S-docosatriene (NPD1)), a DHA-derived messenger endogenously synthesized by RPE cells, is a promoter of RPE cell survival through modulation of the expression of Bcl-2 family proteins and inhibition of effector caspase-3 activity and DNA degradation. In addition, NPD1 potently counteracted cytokine-triggered COX-2 gene promoter induction, another major factor in cell damage. RPE cell damage and apoptosis in turn impair photoreceptor cell survival and seem to be dominant factors in age-related macular degeneration, and in other retinal degenerations, such as Stargardt's disease. Pro-inflammatory injury of the RPE is also involved in pathologic forms of angiogenesis and proliferative vitreoretinopathy, which occur in several diseases, including diabetic retinopathy. NPD1 can be used to enhance photoreceptor survival in retinal degenerations, including age-related macular degeneration and Stargardt's disease.

Miscellaneous

The term “neuroprotectin D1” or “NPD1” as used herein and in the claims refers to 10,17S-docosatriene, its derivatives and analogs. The terms “derivatives” and “analogs” are understood to be compounds that are similar in structure to 10,17S-docosatriene and that exhibit a qualitatively similar effect to the unmodified 10,17S-docosatriene. Examples of such derivatives and analogs can be found in International Application No. WO 04/014835.

The term “therapeutically effective amount” as used herein refers to an amount of docosahexaenoic acid (DHA) or neuroprotectin D1 (NPD1; 10,17S-docosatriene) sufficient to protect a cell (e.g., neural or retinal pigment epithelial (RPE) cell) from oxidative stress or other damage to a statistically significant degree (p<0.05). The term “therapeutically effective amount” therefore includes, for example, an amount sufficient to prevent the degeneration of retinal pigment epithelial cells as found in diseases of age-related macular degeneration or Stargardt's disease, or the neuronal damage due to ischemic stroke, or the symptoms of Alzheimer's Disease by at least 50%. The dosage ranges for the administration of DHA or NPD1 are those that produce the desired effect. Generally, the dosage will vary with the age and condition of the patient. A person of ordinary skill in the art, given the teachings of the present specification, may readily determine suitable dosage ranges. The dosage can be adjusted by the individual physician in the event of any contraindications. In any event, the effectiveness of treatment can be determined by monitoring the degeneration of cells by methods well known to those in the field. Moreover, DHA or NPD1 can be applied in pharmaceutically acceptable carriers known in the art. The application can be oral, topical, by injection, or by infusion.

DHA or NPD1 may be administered to a patient by any suitable means, including orally, parenteral, subcutaneous, intrapulmonary, topically, and intranasal administration. Parenteral infusions include intramuscular, intravenous, intraarterial, or intraperitoneal administration. NPD1 may also be administered transdermally, for example in the form of a slow-release subcutaneous implant, or orally in the form of capsules, powders, or granules.

Pharmaceutically acceptable carrier preparations for parenteral administration include sterile, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. DHA or NPD1 may be mixed with excipients that are pharmaceutically acceptable and are compatible with the active ingredient. Suitable excipients include water, saline, dextrose, glycerol and ethanol, or combinations thereof. Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like.

The form may vary depending upon the route of administration. For example, compositions for injection may be provided in the form of an ampule, each containing a unit dose amount, or in the form of a container containing multiple doses.

DHA or NPD1 may be formulated into therapeutic compositions as pharmaceutically acceptable salts. These salts include the acid addition salts formed with inorganic acids such as, for example, hydrochloric or phosphoric acid, or organic acids such as acetic, oxalic, or tartaric acid, and the like. Salts also include those formed from inorganic bases such as, for example, sodium, potassium, ammonium, calcium or ferric hydroxides, and organic bases such as isopropylamine, trimethylamine, histidine, procaine and the like.

Controlled delivery may be achieved by admixing the active ingredient with appropriate macromolecules, for example, polyesters, polyamino acids, polyvinyl pyrrolidone, ethylenevinylacetate, methylcellulose, carboxymethylcellulose, prolamine sulfate, or lactide/glycolide copolymers. The rate of release of DHA or NPD1 may be controlled by altering the concentration of the macromolecule.

Another method for controlling the duration of action comprises incorporating DHA or NPD1 into particles of a polymeric substance such as a polyester, peptide, hydrogel, polylactide/glycolide copolymer, or ethylenevinylacetate copolymers. Alternatively, DHA or NPD1 may be encapsulated in microcapsules prepared, for example, by coacervation techniques or by interfacial polymerization, for example, by the use of hydroxymethylcellulose or gelatin-microcapsules or poly(methylmethacrylate) microcapsules, respectively, or in a colloid drug delivery system. Colloidal dispersion systems include macromolecule complexes, nanocapsules, microspheres, beads, and lipid-based systems including oil-in-water emulsions, micelles, mixed micelles, and liposomes.

The present invention provides a method of preventing, treating, or ameliorating several deleterious cellular responses that lead to disease states, such as age-related macular degeneration, Stargardt's Disease, and Alzheimer's Disease, comprising administering to a subject at risk for said deleterious cellular response or for said disease or to a subject already affected by said disease, a therapeutically effective amount of either DHA or neuroprotectin. The term “ameliorate” refers to a decrease or lessening of the symptoms or sign of the disorder being tested. The symptoms or signs that may be ameliorated include cellular apoptosis, retinal pigment epithelial cell degeneration, production of toxic P amyloid peptides, etc.

The complete disclosures of all references cited in this application are hereby incorporated by reference. Also, incorporated by reference is the complete disclosure of the following documents: V. L. Marcheselli et al., “Novel Docosanoids Inhibit Brain Ischemia-Reperfusion-mediated Leukocyte Infiltration and Pro-Inflammatory Gene Expression,” J. Biol. Chem., vol. 44, pp. 43807-43817 (2003); Erratum in J. Biol. Chem., vol. 278, p. 51974 (2003); P. K. Mukerjee et al., “Neuroprotectin D1: a docosahexaenoic acid-derived docosatriene protects human retinal pigment epithelial cells from oxidative stress,” Proc. Natl. Acad. Sci. (USA), vol. 101, pp. 8491-8496 (2004); and W. J. Lukiw et al., “Neuroprotective and Anti-inflammatory Bioactive Docosahexaenoic Acid Mediators in Alzheimer's Disease, a manuscript in preparation. In the event of an otherwise irreconcilable conflict, however, the present specification shall control.

1. A method of ameliorating, treating or preventing degeneration of retinal pigment epithelial cells in a mammal, said method comprising administering to the mammal a therapeutically effective amount of at least one compound selected from the group consisting of docosahexaenoic acid (DHA) and Neuroprotectin D1.

2. A method as in claim 1, wherein the compound is DHA.

3. A method as in claim 1, wherein the compound is Neuroprotectin D1.

4. A method as in claim 1, wherein the mammal is a human.

5. A method of ameliorating, treating or preventing age-related macular degeneration in a mammal, said method comprising administering to a mammal a therapeutically effective amount of neuroprotectin D1.

6. A method as in claim 5, additionally comprising administering docosahexaenoic acid (DHA).

7. A method as in claim 5, wherein said mammal is a human.

8. A method of ameliorating, treating or preventing Stargardt's disease in a human, said method comprising administering to a human a therapeutically effective amount of neuroprotectin D1.

9. A method as in claim 8, additionally comprising administering docosahexaenoic acid (DHA).

10. A method of ameliorating, treating or preventing damage to retinal pigment epithelial cells in a mammal that would otherwise be caused by oxidative stress, said method comprising administering to the mammal a therapeutically effective amount of at least one compound selected from the group consisting of docosahexaenoic acid (DHA) or neuroprotectin D1.

11. The method as in claim 10, wherein the oxidative stress is caused by increased cytokine activation.

12. A method as in claim 10, wherein the compound is DHA.

13. A method as in claim 10, wherein the compound is Neuroprotectin D1.

14. A method as in claim 10, wherein said mammal is a human.

15. A method of ameliorating, treating or preventing a retinal degenerative disease in a mammal caused by N-retinylidine-N-retinylethanolamine accumulation in retinal pigment epithelial cells, said method comprising administering to a human a therapeutically effective amount of at least one compound selected from the group consisting of docosahexaenoic acid (DHA) or neuroprotectin D1.

16. A method as in claim 15, wherein the retinal degenerative disease is Stargardt's disease.

17. A method as in claim 15, wherein the compound is DHA.

18. A method as in claim 15, wherein the compound is Neuroprotectin D1.

19. A method as in claim 15, wherein the mammal is a human.

20. A method of ameliorating, treating or preventing apopotosis in the cells of a mammal, said method comprising administering to the mammal a therapeutically effective amount of neuroprotectin D1.

21. A method as in claim 20, wherein the cells are neural cells.

22. A method as in claim 20, wherein the cells are retinal pigment epithelial cells.

23. A method as in claim 20, wherein the cellular apopotosis is associated with Alzheimer's Disease.

24. A method as in claim 20, additionally comprising administering docosahexaenoic acid (DHA).

25. A method as in claim 20, wherein the mammal is a human.

26. A method of increasing the concentration of anti-apoptotic proteins in a mammal, said method comprising administering to a human a therapeutically effective amount of neuroprotectin D1.

27. A method as in claim 26, wherein the anti-apoptotic protein is Bcl-2.

28. A method as in claim 26, wherein the anti-apoptotic protein is Bcl-xL.

29. A method as in claim 26, wherein the anti-apoptotic protein is Bfl1(A1).

30. A method as in claim 26, additionally comprising administering docosahexaenoic acid (DHA).

31. A method as in claim 26, wherein the mammal is a human.

32. A method of ameliorating, treating or preventing brain damage due to ischemic stroke in a mammal, said method comprising administering to a human a therapeutically effective amount of neuroprotectin D1.

33. A method as in claim 32, additionally comprising administering one or more different compounds selected from the group consisting of aspirin and docosahexaenoic acid (DHA).

34. A method as in claim 32, wherein the mammal is a human.

35. A method of ameliorating, treating or preventing Alzheimer's Disease in a human, said method comprising administering to a human a therapeutically effective amount of neuroprotectin D1.

36. A method as in claim 35, additionally comprising administering docosahexaenoic acid (DHA).

37. A method of ameliorating, treating or preventing cognitive dysfunction in an aging human, said method comprising administering to a human a therapeutically effective amount of neuroprotectin D1.

38. A method as in claim 37, additionally comprising administering docosahexaenoic acid (DHA).
